(19) United States
(12) Patent Application Publication
(10) Pub. No.: US 2024/0285594 A1
(43) Pub. Date: Aug. 29, 2024

(54) COMPOSITIONS AND METHODS FOR TREATING PAIN IN ANIMALS

(71) Applicant: North Carolina State University, Raleigh, NC (US)

(72) Inventors: Kristen M. Messenger, Raleigh, NC (US); Emily Wiloch, Raleigh, NC (US); Lilly Smith, Raleigh, NC (US); Ronald E. Baynes, Raleigh, NC (US); Emma Nixon, Raleigh, NC (US); Hiroko Enomoto, Raleigh, NC (US); Monique Pairis-Garcia, Raleigh, NC (US)

(21) Appl. No.: 18/589,673

(22) Filed: Feb. 28, 2024

Related U.S. Application Data
(60) Provisional application No. 63/487,479, filed on Feb. 28, 2023.

(51) Int. Cl. A61K 31/44; A61K 9/00; A61P 29/00

(52) U.S. Cl. CPC A61K 31/44; A61K 9/0043; A61P 29/00

(57) ABSTRACT

The present disclosure provides compositions and methods related to the treatment and/or reduction of pain in an animal. In particular, the present disclosure provides novel compositions and methods for treating and/or reducing pain in an animal (e.g., a pig) by administering an analgesic intranasally. The composition and methods described herein provide a more convenient and effective means for treating and/or reducing pain in an animal (e.g., pain associated with industrial processing) in a manner that also improves the animal's health and welfare.

### CROSS-REFERENCE TO RELATED APPLICATIONS

This application claims the benefit of U.S. Provisional Application No. 63/487,479, filed Feb. 28, 2023, the content of which is herein incorporated by reference in its entirety.

### GOVERNMENT FUNDING

This invention was made with government support under grant number 2019-41480-30292 awarded by the National Institute of Food and Agriculture. The government has certain rights in the invention.

### FIELD

The present disclosure provides compositions and methods related to the treatment and/or reduction of pain in an animal. In particular, the present disclosure provides novel compositions and methods for treating and/or reducing pain in an animal (e.g., a pig) by administering an analgesic intranasally. The composition and methods described herein provide a more convenient and effective means for treating and/or reducing pain in an animal (e.g., pain associated with industrial processing) in a manner that also improves the animal's health and welfare.

### BACKGROUND

Approximately 134 million piglets are produced in the United States each year (USDA Quarterly Hogs and Pigs, March 2020), and a vast majority of these piglets undergo painful husbandry procedures such as castration and tail-docking. Legislation in the European Union and Canada requires that piglets receive anesthetic and/or analgesic drugs for painful procedures, including castration. However, in the United States, piglets undergo these procedures without pain relief as there are currently no FDA-approved analgesic drugs for swine, and there are economic barriers to the practicality of analgesics. Ideally, an analgesic treatment for piglets will be safe and effective, easy to administer by farm personnel, low cost, and long-acting, which leaves few feasible therapeutic options.

### SUMMARY

Embodiments of the present disclosure include a method of treating and/or reducing pain in an animal. In accordance with these embodiments, the method includes administering at least one dose of a composition comprising an analgesic to the animal, wherein the at least one dose is administered intranasally.

In some embodiments, the at least one dose is administered to the animal prior to, concomitant with, or after performing a procedure on the animal, and wherein the administration reduces pain in the animal caused by the procedure. In some embodiments, the procedure comprises one or more of castration, dehorning/disbudding, ear notching, branding, teeth clipping, tail docking, and other surgical procedures associated with tissue damage.

In some embodiments, the pain reduction in the animal is associated with one or more of a reduction in cortisol levels, a reduction in prostaglandin E2 (PGE₂), and/or a change in activity or behavior of the animal. In some embodiments, the at least one dose is administered to the animal to treat pain. In some embodiments, the pain is caused by a surgical procedure, a processing procedure, an injury, an inflammatory condition, a respiratory disease, and/or an infection.

In some embodiments, the method further comprises administering at least one second dose of an analgesic. In some embodiments, the at least one second dose is administered intranasally. In some embodiments, the at least one second dose is administered intramuscularly, intravenously, transdermally, or orally. In some embodiments, the method further comprises intranasal administration of at least a second dose of the composition comprising an analgesic.

In some embodiments, the intranasal administration comprises use of a spraying, nebulizing, or atomizing device.

In some embodiments, the analgesic is a non-steroidal anti-inflammatory drugs (NSAIDs) drug. In some embodiments, the NSAID is selected from the group consisting of flunixin, ketoprofen, and meloxicam, or a pharmaceutically acceptable salt thereof. In some embodiments, the NSAID is flunixin, or a pharmaceutically acceptable salt thereof.

In some embodiments, the composition is administered at a dose ranging from about 0.1 mg/kg to about 10 mg/kg of the analgesic. In some embodiments, the composition is administered at a dose ranging from about 1 mg/kg to about 5 mg/kg of the analgesic.

In some embodiments, the animal is selected from the group consisting of horses, cows, pigs, sheep, goats, cats, and dogs. In some embodiments, the animal is a pig. In some embodiments, the pig is a neonatal pig. In some embodiments, the pig is at least 3 days old. In some embodiments, the animal is a cow.

### BRIEF DESCRIPTION OF THE DRAWINGS

FIG. 1: Treatment allocation for part I. Procedure: surgical castration or sham castration; treatment: physiological saline or buffered lidocaine and/or flunixin (2.2 mg/kg); route of administration: Intramuscular (IM) and/or Intranasal (IN).

FIG. 2: Flow chart of the study (Part I) design based on hour relative to castration. Litters enrolled in the study were randomly assigned to: (1) surgical castration or sham castration; (2) treatment: physiological saline or buffered lidocaine and/or flunixin (2.2 mg/kg); (3) route of administration: Intramuscular (IM) and/or Intranasal (IN).

FIG. 3: Flow chart of the study design (Part II) based on hour relative to castration. Litters enrolled in the study were randomly assigned to: T1: (C) Castration+physiological saline (IM & IN) n=30 and T2: (CLF) Castration+buffered lidocaine 2% (IM) Flunixin (IN) n=29.

FIG. 4: Boxplot of cortisol concentrations (pg/ml) for piglets in the C, S, CL, CF, SF, SL, CLF and SLF groups (T1: (C) Castration plus physiological saline (IM and IN; n=25); T2: (S) Sham plus physiological saline (IM and IN; n=25); T3: (CL) Castration plus buffered lidocaine (IM; n=25); T4: (SL) Sham plus buffered lidocaine (IM; n=25); T5: (CF) Castration plus flunixin (IN; n=25); T6: (SF) Sham plus flunixin (IN; n=24); T7: (CLF) Castration plus buffered lidocaine (IM) and flunixin (IN; n=24); T8: (SLF) Sham plus buffered lidocaine (IM) and flunixin (IN; n=24)) over three timepoints. Timepoint (P<0.01), treatment (P<0.01) and treatment by timepoint (P<0.01) effect. Symbols: circle • indicates outliers; diamond ♦ indicates the mean. Different capital letters show differences statistically significant (P<0.05) where A>B>C>D.

FIG. 5: Boxplots of UPAPS (Unesp-Botucatu Pig Acute Pain Scale) for piglets in the C and CLF groups over three timepoints. Timepoint (P<0.01), treatment (P<0.01) and treatment by timepoint (P<0.01) effect. Symbols: circle (•) indicates outliers; diamond (♦) indicates the mean; the horizontal gray dashed line indicates the UPAPS's optimal cut-off point (≥4). Different capital letters show differences statistically significant (P≤0.05) where A>B>C>D.

FIG. 6: Flunixin plasma concentration (mean, pg/mL) versus time (minutes) after 3.0 mg/kg intranasal administration of flunixin meglumine in one grower pig of various catheter placements, including jugular vein, auricular artery, and femoral artery. This was a part of the pilot study to determine if jugular venous catheter placement yielded similar results.

FIG. 7: Flunixin plasma concentration (mean±standard deviation, μg/mL) versus time (hours) after intranasal (IN) and intramuscular (IM) administration of 2.2 mg/kg flunixin meglumine in six grower pigs. The limit of quantification was 0.001 μg/mL and 0.1 μg/mL for samples in the range of 0.0001-0.1 μg/mL and 0.05-5 μg/mL, respectively.

FIG. 8: Flunixin plasma concentration (mean±standard deviation, μg/mL) versus time (hours) of six pigs for the first 4 hours following administration of 2.2 mg/kg of intranasal (IN) and intramuscular (IM) flunixin. The limit of quantification was 0.001 μg/mL and 0.1 μg/mL for samples in the range of 0.0001-0.1 μg/mL and 0.05-5 μg/mL, respectively. The dot line indicates 0.06 μg/mL of therapeutic level (Nixon et al., 2022).

FIG. 9: Mean±SEM prostaglandin E₂⁻ (PGE₂) concentrations in male piglets that underwent (1) intra-inguinal (IG) and intranasal (IN) physiological saline administration followed by surgical castration (C; n=24), (2) IG and IN physiological saline administration followed by sham castration (S; n=25), (3) IG lidocaine administration followed by surgical castration (20 mg/kg; CL; n=24), (4) IG lidocaine administration followed by sham castration (20 mg/kg; SL; n=25), (5) IN flunixin meglumine administration followed by surgical castration (2.2 mg/kg; CF; n=25), (6) IN flunixin meglumine administration followed by sham castration, (2.2 mg/kg; SF; n=24), (7) IG lidocaine (20 mg/kg IG) and IN flunixin meglumine (2.2 mg/kg IN) administration followed by surgical castration (CLF, n=24), or (8) IG lidocaine (20 mg/kg IG) and IN flunixin meglumine (2.2 mg/kg IN) administration followed by sham castration (SLF; n=24). Pairwise comparisons of treatments at −24 h: P≥0.77. Pairwise comparison of treatments at 1 h: S vs SF−P=0.12, SL vs SLF−P=0.009, C vs CF−P≤0.0001, CL vs CLF−P≤0.0001, SL vs SF−P=0.007, CL vs CF−P≤0.0001). Pairwise comparisons of treatments at 24 h post-castration: P≥0.11.

FIG. 10: Plasma concentration versus time curve for individual cattle (n=4) following a single intravenous dose of flunixin meglumine at 2.2 mg/kg. Samples were simultaneously collected from the opposite jugular vein (via pre-placed catheter) and a pre-placed portal vein catheter. Some variability is observed between sampling sites (jugular vs. portal vein) but overall concentrations and trend are similar over time.

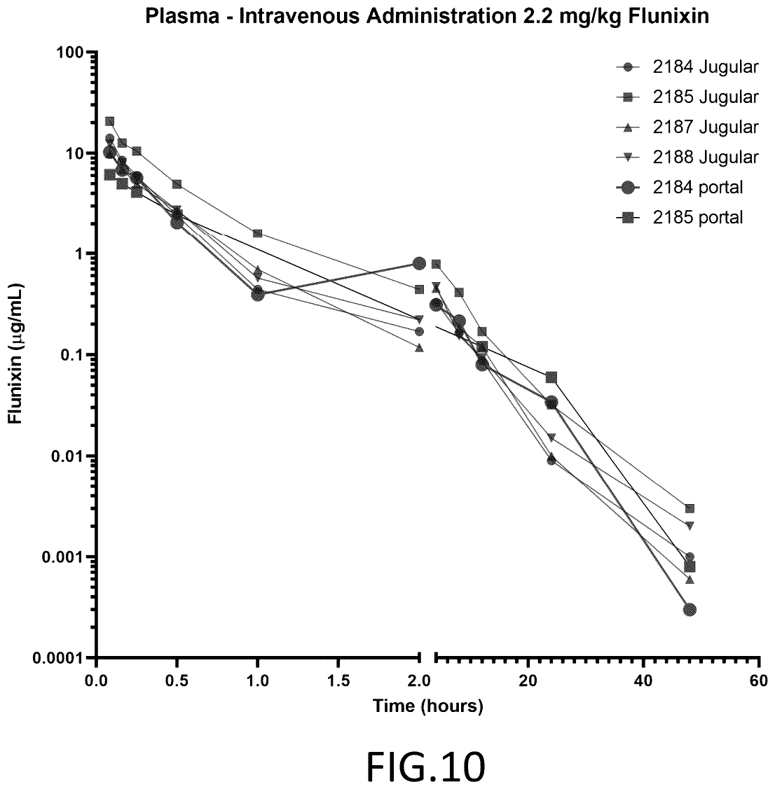

FIG. 11: Plasma concentration versus time curve for individual cattle (n=4) following a single intranasal dose of flunixin meglumine at 2.2 mg/kg. Samples were simultaneously collected from a jugular vein catheter and a pre-placed portal vein catheter. Overall, plasma concentrations are lower following intranasal administration, and the jugular sampling site overestimates systemic concentrations especially following the first 1 hour after administration.

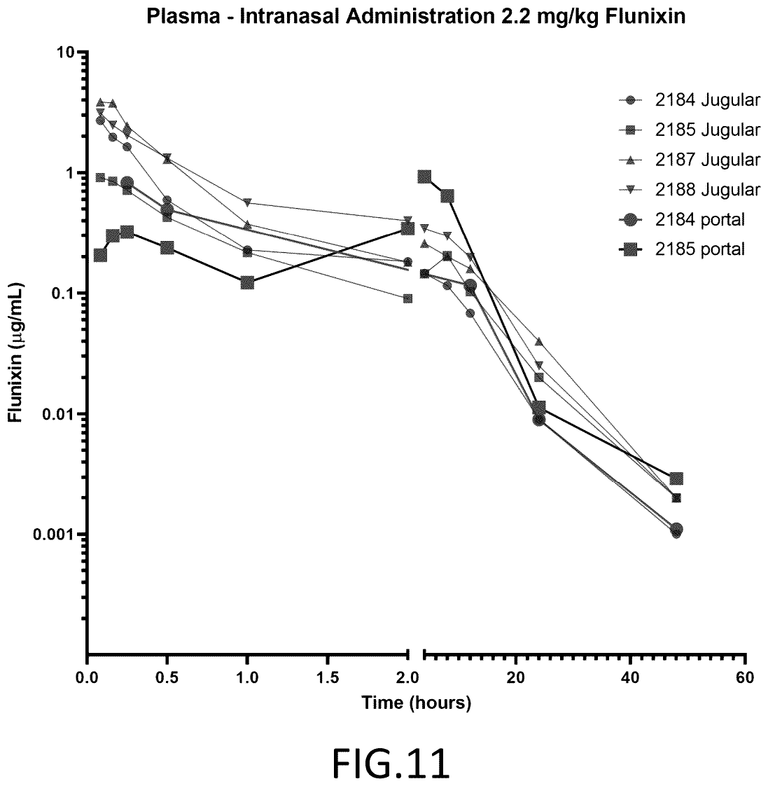

FIG. 12: Interstitial fluid concentration versus time curve for individual cattle (n=4) following a single intranasal (n=2) or intravenous (n=2) dose of flunixin meglumine at 2.2 mg/kg. Samples were collected using an in vivo ultrafiltration collection device and represent tissue unbound concentrations of flunixin. There is systemic absorption following both intravenous and intranasal routes of administration, with tissue concentrations of flunixin detected for at least 24 hours following a single dose.

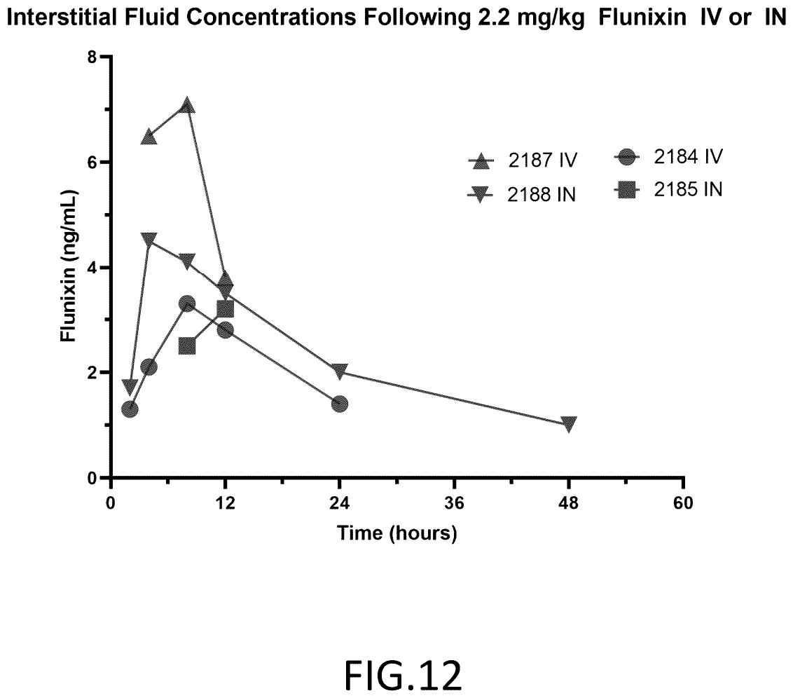

### DETAILED DESCRIPTION

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, the term “animal” refers to any animal (e.g., a mammal), including, but not limited to, humans, non-human primates, pigs, rodents (e.g., mice, rats, etc.), flies, and the like. As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

The terms “administration of” and “administering” with respect to the compositions described herein generally refers to providing a composition of the present disclosure to an animal in need of treatment (e.g., treating and/or preventing pain). In some embodiments, the compositions can be administered by injection (e.g., intramuscular injection) into the animal or by direct application of the composition to a portion of the animal's body (e.g., intranasal administration). In some embodiments, the composition is formulated as a medicament that is applied directly to a portion of an animal's body (e.g., topical application). Routes of systemic administration are also possible, in accordance with the compositions and methods described herein.

As used herein, “intranasal administration” or “administered intranasally” refers to delivery to the nose, nasal passageways or nasal cavity by spray, drops, powder, gel, inhalant or other means.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier and/or excipient. When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

The term “pharmaceutically acceptable carrier, excipient, or vehicle” as used herein refers to a medium which does not interfere with the effectiveness or activity of an active ingredient and which is not toxic to the hosts to which it is administered and which is approved by a regulatory agency of the Federal or a state government or listed in the U.S. Pharmacopeia or other generally recognized pharmacopeia for use in animals, and more particularly in humans. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to saline, buffered saline, dextrose, water, glycerol, ethanol, and combinations thereof. The use of such media and agents for an active substance is well known in the art.

As used herein, the term “effective amount” generally means that amount of a drug or pharmaceutical agent that will elicit the biological or medical response of a tissue, system, animal or human that is being sought, for instance, by a researcher or clinician. Furthermore, the term “therapeutically effective amount” generally means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The term “combination” and derivatives thereof, as used herein, generally means either, simultaneous administration or any manner of separate sequential administration of a therapeutically effective amount of Compound A, or a pharmaceutically acceptable salt thereof, and Compound B or a pharmaceutically acceptable salt thereof, in the same composition or different compositions. If the administration is not simultaneous, the compounds are administered in a close time proximity to each other. Furthermore, it does not matter if the compounds are administered in the same dosage form (e.g., one compound may be administered topically and the other compound may be administered orally).

As used herein, the term “treat,” “treating,” or “treatment” are each used interchangeably herein to describe reversing, alleviating, or inhibiting the progress of a disease and/or injury, or one or more symptoms of such disease, to which such term applies, and/or to improve/enhance one or more aspects of a subject's physical health (e.g., treat and/or prevent pain). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

As used herein, the term “salts” and “pharmaceutically acceptable salts” generally refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disuSfonic, oxalic, and isethionic, and the like. Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, isopropanol, and the like. Lists of suitable salts can be found, for example, in Remington's Pharmaceutical Sciences, 17th ed., Mack Publishing Company, Easton, Pa., 985.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Methods of Treatment

Embodiments of the present disclosure provide compositions and methods related to the treatment and/or reduction of pain in an animal. In particular, the present disclosure provides novel compositions and methods for treating and/or reducing pain in an animal (e.g., a pig) by administering an analgesic intranasally. The composition and methods described herein provide a more convenient and effective means for treating and/or reducing pain in an animal (e.g., pain associated with industrial processing) in a manner that also improves the animal's health and welfare.

Ideally, an analgesic treatment for an animal (e.g., piglet) will be safe and effective, easy to administer by farm personnel, low cost, and long-acting, which leaves few feasible therapeutic options. For example, nonsteroidal anti-inflammatory drugs (NSAIDs) are approved in Europe and Canada to reduce pain associated with piglet castration, but require an intramuscular injection for administration. There are disadvantages to providing analgesia by intramuscular injection in swine: 1) possible pain associated with the injection; 2) broken needles can result in residual needle fragments in pork carcasses; 3) risk of injury or abscesses at the injection site; 4) risk of accidental worker self-injections; 5) requirements for safe needle disposal.

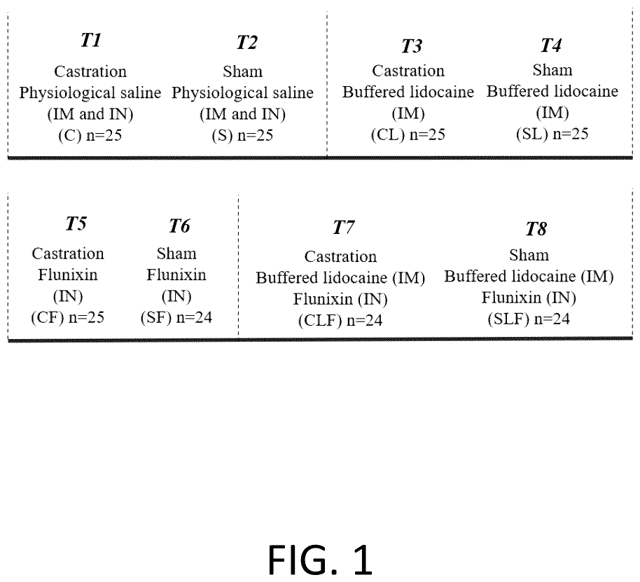

In the US, for example, flunixin meglumine is an NSAID approved via the intramuscular route in swine for treatment of pyrexia caused by respiratory disease. Flunixin is commonly used in an extra-label manner to treat pain, as the Animal Medicinal Drug Use Clarification Act allows veterinarians to prescribe extra-label medications for the improvement of animal health and welfare. As described further herein, embodiments of the present disclosure include methods and compositions for intranasal administration of flunixin meglumine that can be quickly and easily used on farm to treat pain in pigs and improve animal welfare. Intranasal administration has many benefits as a non-invasive route of administration. Unlike intramuscular flunixin administration, intranasal administration does not require the use of needles. This increases worker safety, eliminates the change of muscle injury or needles found in the carcass, and minimizes animal discomfort during administration. A transdermal formulation of flunixin is available for cattle, however this formulation has low bioavailability in pigs meaning it is likely not suitable for pain relief in this species. Intranasal flunixin displays a much better absorption profile, as shown in FIG. 1, and is likely a more suitable non-invasive route of administration.

As described further herein, piglet husbandry procedures such as castration, tail-docking, teeth clipping, ear notching/tagging, and injections are collectively referred to as “processing” piglets. Commercial farms routinely perform processing procedures without anesthesia or analgesia, even though these procedures are painful and distressing to piglets. Generally, castration and tail-docking procedures have the potential to cause the most detrimental health and welfare effects to an animal.

Surgical castration generally refers to surgical removal of the testicles or destruction of testicular formation. Surgical castration of male piglets is common in many countries, and most male piglets in the United States are castrated. Piglet castration occurs before weaning, most commonly within the first three days of life. There are two main techniques: either two vertical cuts (most common) or one horizontal cut to the skin of the scrotum, and the testes are removed by cutting the spermatic cord with a scalpel or pulling until the cord tears. A new experimental approach proposes using a CO₂ surgical laser rather than a scalpel to reduce pain and inflammation and improve healing time; however, this technique requires further optimization before being clinically applicable. Castration is performed to avoid boar taint in the meat of sexually mature male pigs. Boar taint is the accumulation of two main lipophilic compounds, skatole and androstenone, which cause an offensive smell and taste in meat from intact male pigs. Barrows can be raised beyond puberty without developing strong boar taint; however, they have less efficient feed conversion and more fat deposits than boars. In addition to reducing the risk of boar taint, barrows exhibit less sexual and aggressive behavior, making them easier to handle and less likely to fight and injure each other in group pens.

Surgical castration of piglets is painful and distressing with potential adverse health impacts as demonstrated by changes in intensity and frequency of vocalizations, increased pre-weaning mortality, changes in behavior, increased heart rate, and increased cortisol levels. While painful responses are seen up to 4 days post-castration, there is limited information regarding possible chronic pain beyond that time frame. Prolonged pain negatively affects immune function and growth in pigs, and early life pain and stress in other species has significant long-term effects on pain processing, health outcomes, and susceptibility to chronic pain.

Currently, the only viable alternatives to surgical castration are raising intact pigs or immunocastration. As mentioned previously, downsides associated with raising intact pigs can include aggression between pigs and caretakers and the development of boar taint. Immunocastration is an active immunization against gonadotropin-releasing hormone (GnRH). The vaccine consists of a GnRH construct that elicits anti-GnRH antibody production and, therefore, prevents stimulation of luteinizing hormone and follicle-stimulating hormone. This results in suppression of testicular development and steroid production in Leydig cells. The first of this series of two vaccines is administered around 8-12 weeks of age, and the second vaccination is around 4-6 weeks before slaughter. Within a few days of the second vaccination, the boars behave like castrates (reduced aggression and mounting behavior, increased feed intake), and the levels of androsterone/skatole are low. Three vaccinations are required in production systems where the animals are slaughtered at a heavy weight. The main disadvantage to immunocastration is the cost of labor and the vaccine itself, and particularly the second (or third) vaccination can be more difficult in large animals. Monitoring for non-responders after the second vaccine is also necessary based on behavior and size of the testes, there is a health risk for the workers who administer the vaccine due to the risk of self-injection, and consumer perception and acceptance is unclear. Despite these difficulties, there may be some economic benefit associated with immunocastration. Before the second vaccination, immunocastrated pigs are biologically like intact males, exhibiting a more efficient feed conversion ratio and growth than surgically castrated pigs. After the second vaccination, the feed intake of immunocastrated pigs increases, but they still exhibit more efficient growth than surgically castrated pigs.

Additionally, amputation of the distal portion of the tail is intended to reduce the prevalence of tail biting, an abnormal behavior that may result in injury, inflammation, reduced weight gain, increased risk of infection, and even necessitating euthanasia. Tail biting can lead to considerable economic loss to producers and has implications for poor animal welfare. Tail biting is a multifactorial syndrome, and many internal and external factors may affect the prevalence, such as weaning age, diet, genetics, gender, health status, climate, ventilation, stocking density, lack of stimuli, and other environmental factors. To address tail-biting, there have been developments in environmental enrichment strategies, alternative housing systems, and precision livestock farming; however, there has been a failure in applying these findings on commercial farms. Despite EU legislation stating that routine tail docking is forbidden and may only be performed when there is evidence that tail biting has occurred, research suggests that 81%-100% of EU pigs are tail-docked routinely. Therefore, implementation of pain management at tail-docking is critical.

The tail is sensitive and innervated, and tail docking causes both physiological and behavioral responses. Acute responses include increased blood cortisol concentrations, changes in white blood cell count, increased intensity or duration of vocalizations, changes in ear posture, increased dog-sitting and scooting behavior, increased tail-jamming, increased time spent lying alone, increased time spent away from the sow and decreased mechanical nociceptive thresholds. Tail docking is traditionally performed using side cutter pliers or with gas-heated cautery clippers. However, tail docking may also lead to the development of neuromas associated with increased sensitivity to pain regardless of method. Other long-term changes include changes in tail posture, hesitancy to interact with an unknown immobile human (possibly fear of humans), hypersensitivity determined via decreased mechanical nociceptive thresholds, as well as sustained transcriptomic expression changes in caudal dorsal root ganglia cells involved in inflammatory and neuropathic pain pathways.

Individual piglet identification is becoming increasingly important as meat safety and traceability issues increase. The most commonly used methods of identification include ear notching, ear tagging, and tattooing. Implantation of transponders is also an option; however, this is often cost-prohibitive. These methods have associated problems, such as loss of tags, difficulty reading tattoos, and time and labor to apply notches or migration of transponders. Both ear notching and ear tagging are painful; notching elicits vocalizations with high peak frequency, while tagging elicits vocalizations with high mean frequency. Significant increases in head-shaking are also associated with both ear tagging and notching. There is limited research into the welfare effects of tattooing in pigs, but this method does cause increased cortisol and stress in pigs.

Pain is a complex multi-dimensional experience involving both sensory and affective components. It is difficult to assess in veterinary species, and studies often require proxy or indirect measures to quantify pain. When taken in isolation, these measures may not be considered as definitive evidence of “pain”. However, when taken together, they may provide evidence of the underlying affective state. To date, piglet pain is generally assessed using either one or multiple of three approaches: Performance measures; Physiological measures; and Behavioral measures.

Several studies of piglet castration have assessed weight gain; however, there is conflicting evidence to support whether castration improves, reduces, or has no effect on short-term weight gain. One study that reports temporary weight loss suggested that processing may occur during the time at which teat order is established; therefore, male castrates may obtain a less productive teat. Alternatively, activity levels may influence the efficiency of suckling, and if piglets suckle less vigorously, the milk yield of the teat may be affected. On the other hand, weight gain could occur as painful piglets are more likely to have reduced activity, reducing calorie expenditure and possibly increasing weight gain. Given the conflicting data, and weight gain or loss is not specific to pain; this may not be a useful measure for determining pain in piglets. However, castration can also increase pre-weaning mortality in low body weight piglets, possibly related to post-procedural complications. While not a measure of pain, increased pre-weaning mortality is a welfare issue of concern and leads to production losses for producers.

Major pathways activated by stressors are the hypothalamic-pituitary-adrenal (HPA) axis and the autonomic nervous system (ANS). Activation of these pathways triggers release of endogenous compounds, including glucocorticoids, catecholamines, and opiate neuropeptides, to promote recovery by increasing metabolism and reducing inflammation. However, sample collection to obtain information regarding these physiological biomarkers typically requires handling or restraining the animal, which can become a source of stress, confounding the results. Despite this limitation, physiological measures are objective and commonly used research of pain associated with piglet castration and tail-docking studies.

Both physical and psychological stressors activate the HPA axis, stimulating the release of corticotrophin-releasing hormone (CRH) from the hypothalamus, which promotes secretion of adrenocorticotrophic hormone (ACTH) from the anterior pituitary. ACTH then acts on the adrenal cortex to produce cortisol. However, this response is not pain-specific. Tissue trauma associated with surgery can lead to increased cortisol and ACTH even under general anesthesia.

Many studies in pigs have examined cortisol or ACTH concentrations to measure HPA axis activity relating to processing pain. A majority of studies show that castration increases cortisol and/or ACTH levels after castration, however, the cortisol response to tail-docking seems variable. There are also differences in the timing and degree of increase. First, there is a 40-fold increase in plasma ACTH which peaks within 5 minutes after castration, followed by a 3-fold increase in plasma cortisol which peaks 15-30 minutes after castration. Release of cortisol from the adrenal cortex also promotes the mobilization of glycogen, leading to a transient increase in glucose and lactate. While increased plasma lactate has been observed following piglet castration, no significant changes in blood glucose levels have been found. The authors suggest that this is due to a lack of hepatic glycogen stores in neonatal piglets.

Inflammation is an immediate response to injury or infection, characterized by redness, swelling, heat, pain, and loss of function, and is associated with the acute-phase response, which causes changes in acute-phase proteins (APPs) such as haptoglobin (Hp), C-reactive protein (CRP), and serum amyloid A (SAA). These changes are triggered by pro-inflammatory cytokines released by injured or infected cells. Cytokines, such as tumor necrosis factor-alpha (TNF-α) and interleukin 1 beta (IL1-β), and the previously mentioned acute-phase proteins have been measured with tail docking (183) and castration. No differences in CRP, SAA, or Hp have been found between castrated piglets and piglets that are only handled. However, there were increases in TNF-α and IL1-β in both groups, possibly associated with the sample collection, or because both groups were previously tail-docked, teeth-clipped, and ear-notched. Regardless of the method, no differences in CRP were detected between docked and non-docked piglets at three weeks post-tail-docking. However, at seven weeks, increased CRP levels were present in non-docked piglets due to injuries associated with tail biting.

Cyclooxygenase enzymes are also upregulated by tissue damage and inflammatory stimuli and catalyze the conversion of arachidonic acid to prostaglandins. Prostaglandins contribute to pain signaling by activating and sensitizing nociceptors, leading to an increase in the magnitude of response to noxious stimulation, and increases in prostaglandin E2 (PGE₂) are detected following castration and tail docking in piglets. However, it is important to note that the magnitude of the inflammatory response and the pain experienced are not necessarily proportional.

Substance P (SP) is a neurotransmitter related to pain perception, and there is a significant increase in SP plasma concentration after castration in cattle. SP is released from damaged nerve fibers when tissue damage occurs; however, piglet castration has not elicited an SP response at piglet castration or tail-docking in two separate studies. Another relatively underexplored measure of the response to painful stimuli in pigs is the expression of c-fos and its protein product (Fos). Many types of physiological events induce expression of c-fos in neurons of the central nervous system, and following piglet castration, there are a greater number of Fos-positive dorsal horn neurons in untreated piglets than piglets treated with a local or general anesthetic.

Behavioral measures are commonly used to quantify pain associated with castration and other processing procedures. However, conflicting results have been reported across studies. There are inconsistencies in the methodology used (different ethograms, continuous vs. scan sampling, pain-related behaviors vs. maintenance behaviors) that could be responsible for these conflicts.

Pain-related behaviors are behaviors that are specific to piglet castration (or other procedure). The duration of these behaviors is short, and the frequency of occurrence is variable. A study comparing the effectiveness of scan sampling methodologies (in which behaviors are recorded at selected time-points) compared to continuous sampling determined that these types of behaviors are easily missed when scan sampling is used as the observation period is limited. Piglets only spend less than 5 or 6% of their time expressing these short pain-related behaviors. For these reasons, continuous sampling methodologies are ideal when monitoring pain-related behaviors in piglets. In addition to these, one study also assessed abnormal walking (walking with a limp, back arch or hind leg stiffness; flopping down on the ground while walking), leg crossing or shaking (crossing or shaking hind legs while standing or sitting; scratching the body or ear with the legs is not included), and head shaking (shaking head vigorously from side to side). Although much research has been focused on pain behaviors associated with castration, some studies have observed pain behaviors after tail docking (tail wagging and jamming, and scooting), teeth clipping (teeth champing), and ear notching (head shaking). In response to tail-docking, tail wagging and tail jamming are seen within the first minute after the procedure.

In accordance with the above, embodiments of the present disclosure provide means for treating and/or preventing pain in an animal, such as a piglet, using a novel method for the intranasal administration of a composition comprising an analgesic. In some embodiments, the method includes administering intranasally at least one dose of a composition comprising an analgesic to the animal in order to treat and/or prevent pain in the animal.

In some embodiments, the at least one dose is administered to the animal prior to performing a procedure on the animal, and wherein the administration reduces pain in the animal caused by the procedure. In some embodiments, the at least one dose is administered to the animal after performing a procedure on the animal, and wherein the administration reduces pain in the animal caused by the procedure. In some embodiments, the procedure includes one or more of castration, dehorning/disbudding, ear notching, branding, teeth clipping, tail docking, and other surgical procedures associated with tissue damage, as described further above.

In some embodiments, pain reduction in the animal is associated with one or more physiological parameters that can be assessed or measured as a means for evaluating the extent of the pain reduction. For example, pain reduction in an animal can be associated with a reduction in cortisol levels, a reduction in prostaglandin E2 (PGE₂), and/or a change in activity of the animal. In some embodiments, the intranasal administration of at least one dose of a composition comprising an analgesic to the animal results in a change in one or more of these physiological parameters. As would be recognized by one of ordinary skill in the art based on the present disclosure, an animal can experience pain from a variety of different sources, and the compositions and methods described herein can treat and/or prevent such pain. In some embodiments, the pain is caused by a surgical procedure, a processing procedure (e.g., tail-docking), an injury, an inflammatory condition, a respiratory disease, and/or an infection.

In accordance with these embodiments, the methods of the present disclosure include intranasal administration of one or more doses of the composition comprising an analgesic. In some embodiments, the composition is administered to the animal intranasally prior to performing a procedure known to cause pain or thought to cause pain in the animal. In some embodiments, the composition is administered to the animal intranasally after performing a procedure known to cause pain or thought to cause pain in the animal. In some embodiments, the composition is administered to the animal intranasally after an assessment has been made that indicates that the animal is or may be in pain. In some embodiments, the composition is administered to the animal intranasally after an assessment has been made that indicates that the animal has an injury or disease. In some embodiments, the composition is administered to the animal intranasally in anticipation that the animal may suffer an injury or disease.

In some embodiments, the composition comprising at least one analgesic is administered intranasally to an animal in a single dose, which is effective in treating and/or preventing pain in the animal. In some embodiments, the composition comprising at least one analgesic is administered intranasally to an animal in multiple doses, which are effective in treating and/or preventing pain in the animal. In some embodiments, the composition comprising at least one analgesic is administered intranasally to an animal in multiple doses that are administered daily, weekly, monthly, or yearly, according to a dosing regimen that effectively treats and/or prevents pain in the animal. In some embodiments, one or more physiological parameters are assessed in the animal before each dose, after each dose, or at any point during a dosing regimen. In some embodiments, the dosing regimen is changed as a result of one or more physiological parameters assessed in the animal.

In some embodiments, the composition comprising the analgesic is administered intranasally to the animal using a spraying, nebulizing, or atomizing device.

The analgesic or composition thereof may be administered intranasally as a powdered or liquid nasal spray, suspension, nose drops, a gel or ointment, through a tube or catheter, by syringe, by nasal tampon or by submucosal infusion. Nasal drug delivery can be carried out using devices including, but not limited to, unit dose containers, pump sprays, droppers, squeeze bottles, airless and preservative-free sprays, nebulizers (devices used to change liquid medication to an aerosol particulate form), atomizers, metered dose inhalers, and pressurized metered dose inhalers.

The analgesic or composition thereof may be administered in the form of an aerosol spray using a pressurized pack or a nebulizer and a suitable propellant such as, chlorofluorocarbons, hydrocarbons, compressed air, nitrogen, or carbon dioxide. An aerosol system requires the propellant to be inert towards the pharmaceutical composition. In the case of a pressurized aerosol, the dosage unit may be controlled by providing a valve to deliver an accurately metered amount.

The analgesic or composition thereof may be administered in the form of a powder can using microspheres delivered by a nasal insufflator device (a device to blow a gas, powder, or vapor into a cavity of the body) or pressurized aerosol canister. The insufflator produces a finely divided cloud of the dry powder or microspheres. The insufflator may be provided with means to ensure administration of a substantially metered amount of the pharmaceutical composition. The powder or microspheres should be administered in a dry, air-dispensable form. The powder or microspheres may be used directly with an insufflator which is provided with a bottle or container for the powder or microspheres. Alternatively, the powder or microspheres may be filled into a capsule such as a gelatin capsule, or other single dose device adapted for nasal administration. The insufflator can have means such as a needle to break open the capsule or other device to provide holes through which jets of the powdery composition can be delivered to the nasal cavity.

Accordingly, the methods comprise administering the analgesic or composition thereof intranasally using a nasal delivery device. The nasal delivery device can include, but is not limited to, unit dose containers, pump sprays, droppers, squeeze bottles, airless and preservative-free sprays, nebulizers, dose inhalers, pressurized dose inhalers, insufflators, atomizers, and bi-directional devices. The nasal delivery device can be metered to administer an accurate effective dosage amount to the nasal cavity. The nasal delivery device can be for single unit delivery or multiple unit delivery.

As would be recognized by one of skill in the art based on the present disclosure, any such device can be used as long as it can effectively administer a dose of the composition intranasally to the animal. In some embodiments, the composition is administered at a dose ranging from about 0.1 mg/kg to about 10 mg/kg. In some embodiments, the composition is administered at a dose ranging from about 0.5 mg/kg to about 10 mg/kg. In some embodiments, the composition is administered at a dose ranging from about 1 mg/kg to about 10 mg/kg. In some embodiments, the composition is administered at a dose ranging from about 2 mg/kg to about 10 mg/kg. In some embodiments, the composition is administered at a dose ranging from about 4 mg/kg to about 10 mg/kg. In some embodiments, the composition is administered at a dose ranging from about 6 mg/kg to about 10 mg/kg. In some embodiments, the composition is administered at a dose ranging from about 8 mg/kg to about 10 mg/kg. In some embodiments, the composition is administered at a dose ranging from about 0.1 mg/kg to about 5 mg/kg. In some embodiments, the composition is administered at a dose ranging from about 0.1 mg/kg to about 2 mg/kg. In some embodiments, the composition is administered at a dose ranging from about 0.1 mg/kg to about 1 mg/kg. In some embodiments, the composition is administered at a dose ranging from about 1 mg/kg to about 5 mg/kg. In some embodiments, the composition is administered at a dose ranging from about 1 mg/kg to about 10 mg/kg. In some embodiments, the composition is administered at a dose ranging from about 2 mg/kg to about 8 mg/kg. In some embodiments, the composition is administered at a dose ranging from about 5 mg/kg to about 10 mg/kg.

In accordance with these embodiments, the analgesic contained in the composition and administered intranasally to an animal is a non-steroidal anti-inflammatory drugs (NSAIDs) drug. In some embodiments, the NSAID is selected from the group consisting of flunixin, ketoprofen, and meloxicam. In some embodiments, the NSAID is flunixin, or a pharmaceutically acceptable salt thereof.

As would be recognized by one of ordinary skill in the art based on the present disclosure, any analgesic can be administered intranasally to an animal using the methods of the present disclosure. Additionally, in some embodiments, the composition comprises more than one analgesic, and/or an additional pharmaceutically active agent. In some embodiments, the composition includes a pharmaceutically acceptable adjuvant and/or excipient. In some embodiments, the composition includes a pharmaceutically acceptable adjuvant and/or excipient suitable for use with administration with a spraying, nebulizing, or atomizing device for intranasal administration.

Although the compositions and methods of the present disclosure include the treatment and/or prevention of pain in pigs and piglets, other animals can also be treated using the compositions and methods provided herein. In some embodiments, the animal includes, but is not limited to horses, cows, pigs, sheep, goats, cats, and dogs. In some embodiments, the animal is a cow. In some embodiments, the animal is a pig. In some embodiments, the pig is a neonatal pig. In some embodiments, the pig is at least 3 days old. In some embodiments, the pig is less than two years old. In some embodiments, the pig is less than a year old. In some embodiments, the pig is less than six months old. In some embodiments, the pig is less than three months old. In some embodiments, the pig is more than a year old.

### 3. Examples

Embodiments of the present disclosure include a novel intranasal route of administration of flunixin meglumine, a nonsteroidal anti-inflammatory drug (NSAID), in pigs. This method could be used within the Animal Medicinal Drug Use Clarification Act (AMDUCA) in an extra-label manner to treat painful conditions in pigs. Examples of painful conditions within the swine industry include piglet processing (castration, tail docking, needle teeth clipping, ear notching) and lameness in older pigs. While an intramuscular administration of flunixin is approved for use in pigs, the intranasal administration is advantageous because it can be easily and quickly given on farms to a large number of animals. This method does not require needles, thus preventing additional pain associated with the injection and preventing accidental self-injection in farm workers administering the drug to pigs. Finally, needles can result in injection site abscesses, which reduces carcass quality. This novel route of flunixin will help fill a need for accessible analgesic options for piglet processing, especially as public concern for food animal welfare has grown in recent years. By showing the public that they are committed to animal welfare by using this method of pain relief during processing, the swine industry could gain more consumer trust and potentially more profit.

Thus, experiments were conducted to test the efficacy of intranasal administration of an analgesic to an animal, in accordance with the embodiments described herein. Flunixin meglumine is a nonsteroidal anti-inflammatory drug (NSAID) approved by the Food and Drug Administration (FDA) for use in cattle, horses, and swine to treat inflammatory conditions. This drug is approved for treatment of pyrexia associated with respiratory tract disease in swine, administered by a single intramuscular (IM) injection. Since there are no approved analgesic drugs for swine, flunixin is often used extra-label to treat pain. While the pharmacokinetics of intravenous, intramuscular, oral, and transdermal administrations of this drug have been studied, no published studies have investigated the use of intranasal (IN) administration. Piglet processing typically includes painful procedures such as tail docking, castration, and clipping needle teeth and is often performed without analgesics. A method of administration that is easily administered on farms and efficiently administered to large numbers of pigs could have a major impact on the animal welfare and the economics of the industry.

The objective of this experiment was to determine whether IN administration of flunixin could be used for pain management in piglets and grower pigs. In these experiments, intranasal delivery was provided via a syringe attached to a laryngo-tracheal mucosal atomization device (LMA MADgic, Teleflex Medical, RTP, NC). However, other delivery devices can also be used for systemic uptake from the nasal mucosa; a drug can be absorbed through highly vascularized nasal mucosa into the bloodstream and circulate systemically through the body.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Managing castration pain on US sow farms is hindered by the lack of Food and Drug Administration (FDA) approved products for mitigating pain. Previous work assessing flunixin meglumine (FM) efficacy in mitigating castration pain has shown the drug to be effective in pigs, meanwhile, results from previous work evaluating lidocaine efficacy are contradictory. Therefore, the objective of this study was to determine the efficacy of buffered lidocaine (BL) and FM in mitigating castration pain in piglets. This study was divided into Part I (physiological response) and Part II (behavioral response). For part I piglets were randomly assigned to the following treatments: T1: (C) Castration plus physiological saline; T2: (S) Sham plus physiological saline; T3: (CL) Castration plus BL; T4: (SL) Sham plus BL; T5: (CF) Castration plus FM; T6: (SF) Sham plus FM; T7: (CLF) Castration plus BL and FM; T8: (SLF) Sham plus BL and FM. Blood was collected 24 h prior to castration, 1 h, and 24 h post castration for cortisol quantification. For Part II another cohort of piglets was enrolled and randomly assign to the following treatments: T1: (C) Castration plus physiological saline and T7: (CLF) Castration plus BL and FM. Behavior scoring was obtained in real-time by observing each piglet for 4-min continuously using Unesp-Botucatu pig acute pain scale (UPAPS) at the following timepoints: 1 h before castration (−1 h), immediately post-castration (0 h), and 3 h post-castration (+3 h). Average cortisol concentrations did not differ at −24 h (P>0.05) or at 24 h post-castration (P>0.05) between treatments. At 1 h post-castration, castrated piglets (C and CL) demonstrated greater cortisol concentrations. Castrated piglets in the CF and CLF group had lower cortisol concentrations compared to C and CL-treated pigs (P<0.05). For behavioral response, there were no differences between treatments on total UPAPS scores (C and CLF, P>0.05). Intranasal FM was able to effectively reduce the physiological piglet's response immediately post-castration. Buffered lidocaine had no effect on the either physiological or behavioral response to pain. Long-term research should focus on refining injection techniques for BL and consider administration frequency and dosing of intranasal FM to control pain for a longer period post-castration.

Castration is a painful procedure performed on piglets around the world. In the US alone, more than 60 million pigs are surgically castrated annually. Castration results in the piglet experiencing acute pain and stress and this procedure negatively impacts farm performance as demonstrated by increases in morbidity and mortality during the pre-wean production period. Managing castration pain on US sow farms is hindered by two main drivers: 1) lack of Food and Drug Administration (FDA) approved products validated for efficacy in mitigating pain and 2) logistical limitations to implementing pain management protocols on a large scale.

In the US, relieving pain in pigs can be prescribed by veterinarians under the Animal Medicinal Drug Use Clarification Act (AMDUCA). This act permits veterinarians to utilize FDA approved products in an extra-label manner (i.e., species and conditions not on the label), thus providing some options for pain relief while the US swine industry awaits approval of pain-specific products for pigs. As opportunities arise to approve products for pain relief, pharmaceutical companies should prioritize products that are effective, easy to administer, require minimal training and are as least invasive as possible, to overcome the logistical limitations found on large commercial farms.

Historically, lidocaine has been used on food animal species to inhibit pain transmission via local anesthesia. Lidocaine works primarily by blocking voltage-gated sodium channels thus inhibiting action potential propagation. Local anesthetics administration prior to castration is required in many European countries including Denmark, where veterinarians train caretakers to administer procaine, making the process more practical. However, results from previous work evaluating lidocaine efficacy for pain mitigation are contradictory. Some work suggests that intra-testicular administration of lidocaine mitigates pain, while other studies indicate that lidocaine does not effectively control post-operative castration pain. In addition, lidocaine administration cannot control pain caused by inflammation from tissue damage during and after the castration process.

Currently in the US, flunixin meglumine (FM) is the most common pain relief used on swine farms. Flunixin meglumine is a non-steroidal anti-inflammatory drug (NSAID) that inhibits cyclooxygenase production and suppresses prostaglandin synthesis. This product can be administered via multiple routes including intramuscular, intravenous, topical, and oral. Previous work assessing FM efficacy in mitigating castration pain has shown the drug to be effective in pigs and other farm animal species undergoing castration.

Transdermal flunixin meglumine was effective in mitigating pain in castrated pigs, suggesting its use as a pharmaceutical option to control pain in large commercial farms given its advantage as a non-invasive, extra-label administration route. To the authors knowledge, no studies to date have evaluated the efficacy of intranasal FM administration in piglets undergoing castration.

Given the great potential of single or multimodal analgesia using FM and lidocaine in mitigating castration pain for swine, further evaluation of the efficacy of both drugs, particularly when administered utilizing less invasive administration techniques, is needed. Therefore, the objective of this study was to determine the efficacy of buffered lidocaine administered intra-inguinally and FM administered intranasally on mitigating castration pain in piglets.

### Materials And Methods.

This was a two-part study completed in the spring of 2022 on a commercial sow farm located in the Southeastern United States. This study was approved by the Institutional Animal Care and Use Committee of North Carolina State University (IACUC protocol 20-113-01). Animals were cared for and handled in accordance with the Guide for the Care and Use of Agricultural Animals in Research and Teaching. No animals were castrated exclusively for the purposes of this study, the piglets' castration was a regular procedure conducted on the farm, that contributes to the four Rs of animal experimentation (reduce, replace, refine, and respect) and the welfare of pigs.

### Housing and Management.

Piglets were housed with sows on fully slatted, tunnel ventilated farrowing rooms. Room temperature was managed through a computerized control system at 220±1.0° C. for the sow and heat mats for piglets were set to approximately 30-35° C. Within each room, sows and litters were housed in individual farrowing crates (2.5 m×0.7 m) with additional space for piglets (2.5 m×1.3 m) surrounding the crates. Lighting was turned on between 600 h and 1630 h. Feed and water were offered ad libitum to sows and piglets.

This study produced two data sets: one for Part I: physiological response and Part II: for behavioral assessment.

### Part I: Physiological Assessment:

### Treatment.

A total of 197 Large White×Duroc cross male piglets from 35 litters were enrolled in the study (Table 1). Piglets were individually identified using ear tags (Allflex Global Piglet ear tags, Allflex Livestock Intelligence, Madison, WI), weighed and randomly allocated to one of eight treatment groups (FIG. 1).

### Treatment Administration:

### Buffered Lidocaine.

Lidocaine was buffered by mixing 2 ml of 8.4% Sodium Bicarbonate to 20 ml of 2% lidocaine HCl injectable solution to achieve a pH of 6.8 (Lidocaine Hydrochloride, Covetrus, Dublin, Ohio, US). Piglets enrolled in lidocaine treatment groups (CL, SL, CLF, SLF−T3: (CL) Castration plus buffered lidocaine (IM; n=25); T4: (SL) Sham plus buffered lidocaine (IM; n=25); T7: (CLF) Castration plus buffered lidocaine (IM) and flunixin (IN; n=24); T8: (SLF) Sham plus buffered lidocaine (IM) and flunixin (IN; n=24)) were injected with buffered lidocaine approximately 20 min prior to surgical castration. Piglets were held by both rear legs by one caretaker with the abdomen facing the individual administrating treatment. Buffered lidocaine was injected intra-inguinally (Picture 1) by a second caretaker using a ½ inch needle (Ideal® D3 20 Gauge, Neogen, Lansing, MI) inserted into a syringe (Prima Tech® 2 cc Bottle Mount Vaccinator, Prima Tech USA, Kenansville, NC). A total of 1.5 ml of buffered lidocaine per injection site was administered intramuscularly (IM) into each inguinal canal (left and right) at a 40-degree angle 5-7 cm from the scrotum and 2-3 cm from the abdominal wall. Piglets enrolled in the control treatment (C, S, CF, SF−T1: (C) Castration plus physiological saline (IM and IN; n=25); T2: (S) Sham plus physiological saline (IM and IN; n=25); T5: (CF) Castration plus flunixin (IN; n=25); T6: (SF) Sham plus flunixin (IN; n=24)) were handled in an identical manner and 1.5 ml of sterile saline was injected in the same two locations as described previously.

### Flunixin Meglumine.

Immediately following intra-inguinal injection, piglets enrolled in the FM treatment groups were held in sternal recumbency by one individual and 2.2 mg/kg (Banamine®, Merck Animal Health, Madison, NJ, US) was administered in one nostril using was a MAD® nasal intranasal mucosal atomization device (Telefex Incorporated, Wayne, PA, US) attached to a Prima Tech® 0.5 cc bottle mount vaccinator. The same individual administered the treatment by gently holding the piglet's snout using their non-dominant hand to steady the head and administered the drug with the other hand. Piglets in the control group were handled in the same manner in an equivalent volume of 0.2 ml of sterile saline was administered as described above.

### Castration Procedure.

Castration was performed by one trained caretaker from the farm. Piglets were picked up, individually held by both hind legs with head down, and two vertical incisions were made through the skin of the scrotum over each testicle using a scalpel blade. Once the incisions were made, testicles were exposed, spermatic cords cut, and testicles were completely removed by traction. A sham castration was performed to mimic similar handling conditions in which piglets were picked up, held in the same manner, and had pressure applied to the scrotal area by the same individual responsible for castration.

### Blood Sampling.

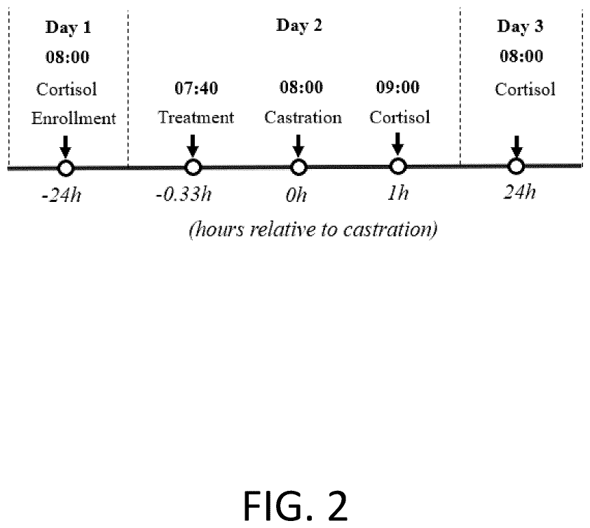

Blood was collected 24 h prior to (−24 h), 1 h (1 h), and 24 h post castration (24 h, FIG. 2). Blood samples were collected using the technique described in other studies. The orbital sinus cavity was punctured using an Excel® disposable hypodermic needle 20G (Exel International, Quebec, Canada) and deposited into a 4 ml BD® red vacutainer serum tube (Med Vet International, Mettawa, IL). All tubes were maintained in a cooler and centrifuged (2,000×g for 15 min at 4° C.) no more than eight hours post-collection to separate serum. Serum was stored in 1.5 ml Axygen® microcentrifuge tubes (Axygen Scientific, Corning, NY) at −80° C. and assays were performed two months later.

### Cortisol Assay.

Serum cortisol concentrations were quantified using a commercially available EIA kit (Arbor Assays DetectX Cortisol EIA Kit, Product #K003). The detection limits of the cortisol assay were 50 pg/ml to 3,200 pg/ml. Samples were diluted 1:100 with assay buffer and run according to kit directions. All samples were assayed in duplicate. In total, forty cortisol assays were performed. Mean intra-assay variation of duplicate samples was 6.7±7.5%. Mean inter-assay variation of two quality control pools was 10.0±0.1% (Merenda et. al., 2022).

### Part II: Behavioral Assessment:

Upon obtaining results from physiological assessment of treatments in Part I, a follow up behavioral study was conducted to assess the efficacy of lidocaine and FM in combination on mitigating castration pain in pigs using a validated piglet pain scale. Another cohort of piglets were enrolled in this second part of this study consisting in a total of 119 Large White×Duroc cross male piglets (60 and 59 piglets for C and CLF respectively, Table 2).

### Behavioral Scoring.

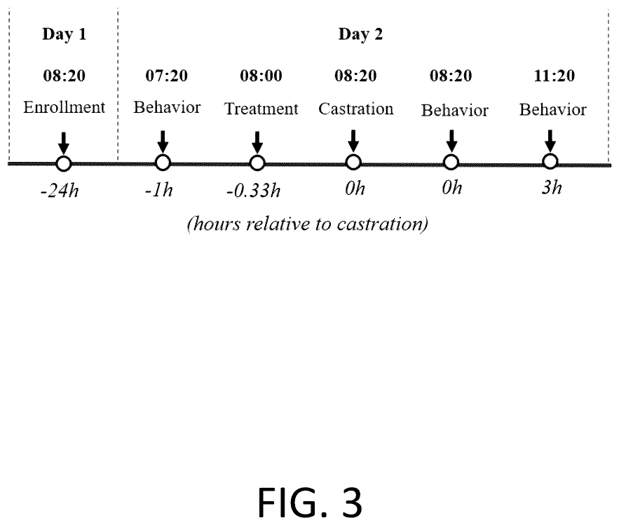

Behavior scoring was obtained in real-time by observing each piglet for 4-min continuously using Unesp-Botucatu pig acute pain scale (UPAPS). Each piglet was scored by one trained observer at the following timepoints: 1 h before castration (−1 h), immediately post-castration (0 h), and 3 h post-castration (+3 h, FIG. 3). The 4-min sampling time was obtained from the methodology previously validated. Treatments were masked, randomized, and applied to each piglet by a senior researcher.

The Unesp-Botucatu UPAPS scale evaluates five behavioral items, with each item divided into four descriptive levels. A numerical score was designated from “0” to “3”, with a “0” representing normal behavior (free of pain) and “3” corresponding to pronounced behavioral deviation (severe pain). Therefore, for each timepoint, piglets may receive a score ranging from 0 (min) to 15 (max; Table 3). Total pain scores were then calculated for each piglet per timepoint.

### Rescue Analgesia.

Following video scoring for each treatment, the observer was required, based on clinical experience, to mark whether the piglet required (yes) or did not require (no) analgesic intervention due to breakthrough pain. This is most referred to rescue analgesia in the literature; and was conducted for each behavioral assessment. Total counts were calculated for piglets identified as requiring or not requiring rescue analgesia by treatment and timepoint.

### Statistical Analysis.

Statistical significance was declared at P≤0.05. All data was analyzed using RStudio (Version 4.1.0; 2021-06-29; RStudio, Inc., Boston, MA, USA, 30).

### Part I:

A multilevel linear model was conducted with the cortisol concentrations after the Box-Cox transformation (k=0.02) to closely reassemble normality attested by Cramer-Von Mises test. Treatments, timepoints (−24 h, 1 h, 24 h), and its interaction were used as fixed effects. Piglet's age, sow parity, and piglet body weight were included as covariables. Piglets nested in the litters were applied as random effects composing each modeling level. The Bonferroni were used for adjustment the P-value to the post-hoc test. Results were illustrated with boxplots using the original cortisol concentration values.

### Part II:

A multilevel generalized linear model adjusted by Poisson distribution was used to analyze total pain score using treatments (C and CLF), timepoints (Baseline at −1 h, immediately post-castration and post-castration at 3 h) and its interaction as fixed effects. Piglet's age and sow parity were included as covariables. Piglets nested in the litters were applied as random effects composing each modeling level. The Bonferroni were used for adjustment after multiple comparisons to the post-hoc test. Results were illustrated with boxplots.

For rescue analgesia based on evaluator clinical experience and based on UPAPS's cutoff point (total sum >4), a test of homogeneity by Chi-square (χ²) was used to determine if the distribution of the piglets in pain requiring rescue analgesia was the same between the two treatments (C and CLF) for each timepoint and the entire period.

### Results, Part I:

Data was collected from a total of 197 male piglets over 35 litters with 5.6±1.7 piglets enrolled per litter. Piglet and litter performance can be found in Table 1.

### Effect of the Treatment and Timepoint on Cortisol Concentrations.

Treatment (P<0.01), timepoint (P<0.01), and the interaction treatment by timepoint (P<0.01) had an effect on cortisol concentrations. Age (P=0.70) and sow parity (P=0.44) had no effect on the cortisol concentration, while the piglet body weight had a negative (β=−0.06) and significant (P≤0.01) effect.

Average cortisol concentrations did not differ at −24 h (P>0.05) or at 24 h post-castration (P>0.05) between treatments. At 1 h post-castration, castrated piglets (C and CL) demonstrated greater cortisol concentrations than piglets assigned to sham treatment groups (S, SF, SL, SLF; P<0.01). Cortisol concentrations between C and CL at 1 hr post-castration were not different (P≥0.05).

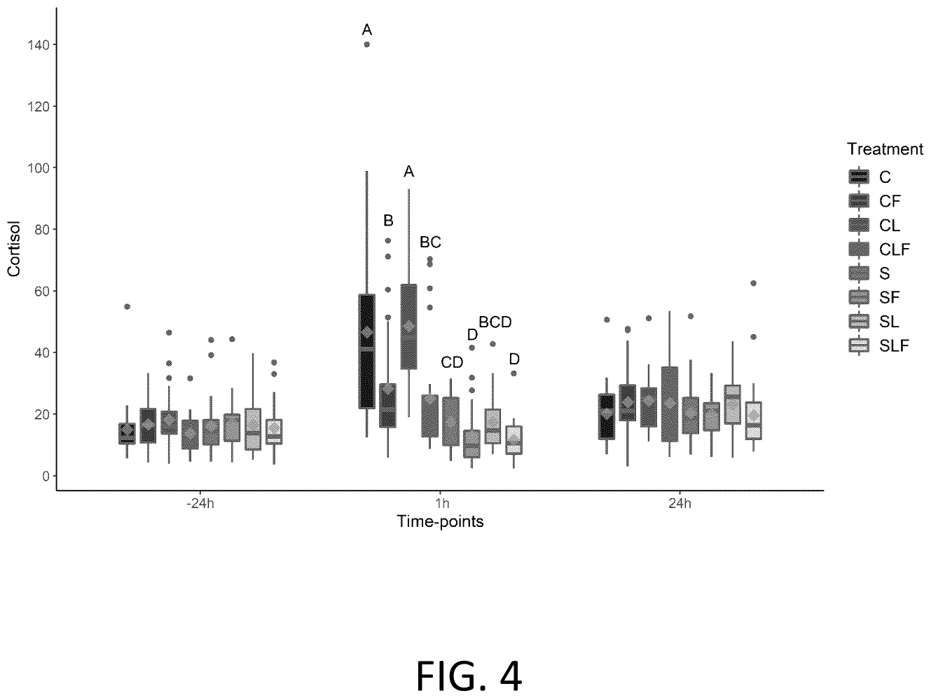

Castrated piglets in the CF and CLF group had lower cortisol concentrations compared to C and CL treated pigs (P<0.05). Sham piglets (S) demonstrated lower cortisol concentrations compared to CF piglets (P<0.05) but were not different compared to CLF treated piglets (P>0.05). Sham piglets treated with FM (SF and SLF) had the lowest cortisol concentrations and were different than all castrated piglets (P<0.01). No differences were found between any sham treatment group at any timepoint (P>0.05, FIG. 4).

### Part II:

Data was collected from 16 litters with a total of 119 male piglets with 3.8±0.8 piglets enrolled per litter. Piglet and litter performance can be found in Table 2.

### Effect of the Drug, Procedure and Timepoint on Total Pain Scores.

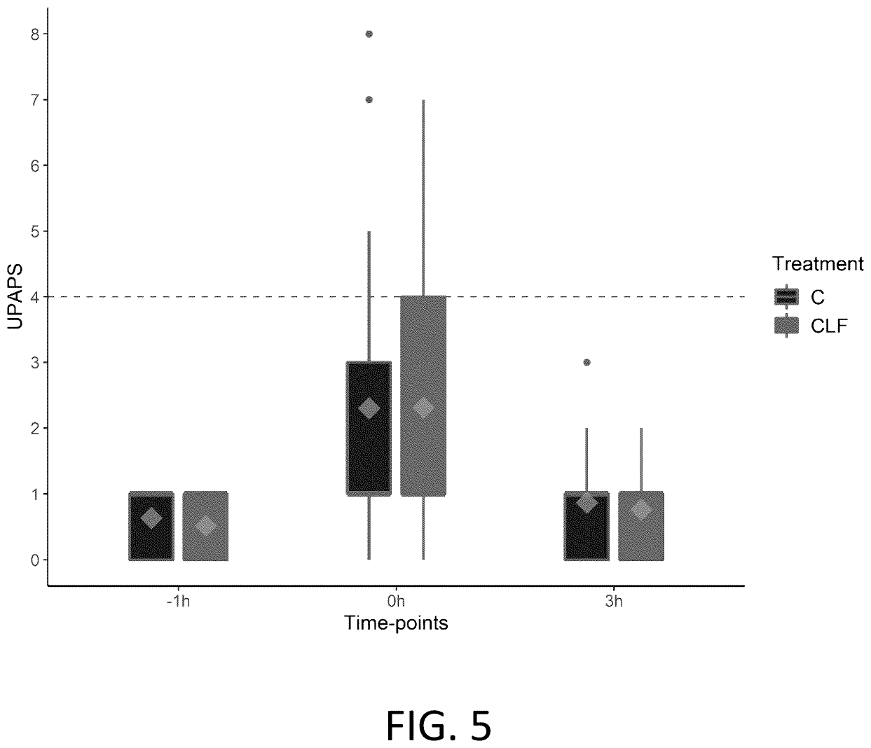

There was a timepoint (P<0.01) effect on UPAPS with total average pain scores greatest immediately post-castration compared to pre-castration timepoint. Piglet age (P>0.05) and sow parity (P>0.05) had no effect on the UPAPS. There were no differences between treatment or treatment by timepoint (P>0.05, FIG. 5).

When assessing rescue analgesic requirement based on evaluator clinical experience, the treatment C (n=90) and CLF (n=97) was not different at timepoint −1 h (C and CLF respectively 0 vs 0; χ²=0.01, P>0.05), 0 h (C and CLF respectively 8 vs 8; χ²=0.00, P>0.05), and 3 h (C and CLF respectively 0 vs 0; χ²=0.01, P>0.05) or in all timepoints (C and CLF respectively 8 vs 8; χ²=0.00, P>0.05).

When assessing rescue analgesic requirement based on UPAPS's cutoff point (total sum ≥4), the treatment C (n=90) and CLF (n=97) was not different at timepoint −1 h (C and CLF respectively 0 vs 0; χ²=0.01, P>0.05), 0 h (C and CLF respectively 7 vs 8; χ²=0.01, P>0.05), and 3 h (C and CLF respectively 0 vs 0; χ²=0.01, P>0.05) or in all timepoints (C and CLF respectively 7 vs 8; χ²=0.01, P>0.05).

Castration is a common procedure performed on farm despite ethical concerns specific to pain experienced by the piglet. Pain mitigation strategies in the US are limited with the majority of work assessing the efficacy of local anesthesia and NSAIDs in controlling castration pain. Pain management protocols should be implemented in a manner that is effective, practical, cost-effective and the least invasive for the piglets. Therefore, the objective of this study was to determine the efficacy of buffered lidocaine administered intra-inguinal and FM administered intranasal on mitigating castration pain in pigs.

The pioneering spirit of the present study was the use of intranasal FM to mitigate castration pain in piglets as demonstrated by decreased cortisol concentrations immediately following castration. This finding agrees with research previously conducted in 2021 by Nixon and colleagues that evaluated intramuscularly administered FM efficacy on castration pain. Results from the 2021 study proved that FM decreased cortisol concentrations 2 h post-castration when compared to a castrated, non-treated control group. In addition, although not significant, cortisol concentrations were also found to numerically decrease by more than 30% in piglets administered FM topically 24 h prior to castration compared to saline-treated piglets. Results from the current study and support from the previously published work suggests that FM's mode of action is effective in mitigating deviations to the physiological response of piglets undergoing castration as determined by decreased cortisol levels immediately following the procedure.

In contrast to the physiological response to castration, piglets administered FM intranasally did not decrease total behavior pain scores and required similar rescue analgesia compared to control piglets. The present work is in direct contrast with previous work that showed transdermal FM administered 24 h before castration decreased total pain scores and rescue analgesia intervention from 54% (control pigs) to 29% (transdermal FM treated pigs). There are several possible explanations for this, including drug absorption variability and behavioral methodology. From a drug absorption standpoint, intranasal administration is often characterized as a rapid route for drug absorption given the nasal mucosa is richly supplied with blood vessels and intranasal administered drugs gain immediate access to systemic circulation. In addition to this, intranasal administered products, as opposed to topically applied products, may bypass the hepatic first-pass effect, thus altering both the concentration and time in which the drug reaches the maximum concentration in the blood. Therefore, moments in which total pain scores and rescue analgesia were assessed in this study may have been influenced by varying absorption time between administration routes thus pain scores may have been assessed when the drug was not at peak efficacy, resulting in non-significant differences between control and treated pigs. Future work must assess pharmacokinetic and pharmacodynamic properties of FM administered intra-nasal to identify Cmax and Tₘₐₓ more effectively for behavioral research.

In addition to absorption variability, behavioral methodology may have also influenced the overall results of this study. The validated pain scale effectively distinguished painful and non-painful states in castrated piglets as observed via deviations in total pain scores across timepoints, however, treatment was not different. In contrast to Lopez-Soriano and colleagues (2022), total pain scores and rescue analgesia were evaluated via live observation as compared to video observation due to farm logistics. Work evaluating piglet behavior has demonstrated that pigs are prey species and will often hide behaviors specific to pain and injury. When comparing total pain scores immediately post castration in this study compared to, it should be noted that total scores were 4.9 for castrated piglets and 3.1 for transdermal flunixin treated piglets in contrast with the present study that the total pain score were approximately 2.3 for both C and CLF. Work conducted in rabbits concluded that the presence of an observer might lead to a false sense of pain. Therefore, future studies should evaluate total pain scores and rescue analgesia utilizing recorded video, thus eliminating the impact of human presence on piglet's pain demonstration.

Buffered lidocaine administered intra-inguinal had no effect on pain mitigation from either a physiological or behavioral standpoint. The results from this study are in agreement with numerous studies that have consistently demonstrated lidocaine does not decrease cortisol concentrations in castrated piglets and in fact may increase cortisol concentrations when compared to castrated piglets receiving no anesthetic. However, past work conducted, and more recent studies in 2022 have demonstrated lidocaine efficacy in mitigating castration pain. There seems to be no consensus in the literature about the effectiveness of lidocaine in reducing behavioral and physiological pain responses in piglets, however, this can be explained by differences in the interval between treatment administration and castration (0, 3, 5, 10 or 20 min). Unlike previous work, this is the first paper utilizing an intra-inguinal approach to administering lidocaine as a local anesthetic, targeting direct inhibition at the spermatic cord. Intra-inguinal injection is not a routine procedure performed on farm and variation exists in injection site location based on pig size, position and individual technique which can be understood as a limitation of the study. Although only one person injected all pigs for the study, it is possible that injection technique was inconsistent, thus resulting in variability of anesthetic efficacy and no difference between piglets castrated with physiological saline (C) and castrated with buffered lidocaine (CL). While buffering the lidocaine provided the advantage of preventing pain associated with the injection site, future studies must consider refining injection technique to ensure spermatic cord innervation is impacted and administration can be consistently given across pigs regardless of size and/or position.

### Animal Welfare Implications and Conclusions.

This research was the first to measure the efficacy of buffered lidocaine administered intra-inguinal in combination with intranasal administered FM. Intranasal FM was able to effectively reduce the physiological response of piglet to castration as demonstrated by decreased cortisol levels immediately post-castration, however behavioral differences were not noted. Hence, from a husbandry view, the implementation of intranasal FM could be an important and feasible step to be applied in large-scale swine farms that normally do not use any drug for pain relief associated with surgical castration.

Buffered lidocaine had no effect on either physiological or behavioral response to pain.

Cortisol concentrations were greater 24 h post-castration compared to baseline concentrations suggesting castrated piglets are still experiencing pain sensitivity one day following castration and a single FM administration was not effective in mitigating post-operative pain. Long-term research projects should focus on refining injection technique for buffered lidocaine and consider administration frequency and dosing of intranasal FM to control pain for a longer period post-castration.

### Tables:

TABLE 1
Mean ± SD. Descriptive statistics for 35
litters at enrollment (Part I; 197 piglets total).

| | |
|---|---|
| Age (days) | 9.0 ± 1.1 |
| Sow parity | 3.9 ± 1.3 |
| Total born | 14.3 ± 1.8 |
| Liveborn | 13.1 ± 1.5 |
| Stillborn | 0.7 ± 0.9 |
| Mummies | 0.5 ± 0.9 |
| Weight (Kg) | 3.2 ± 0.7 |

TABLE 2
Mean ± SD. Descriptive statistics for 16
litters at enrollment (Part II; 119 piglets total).

| | |
|---|---|
| Age (days) | 7.9 ± 0.9 |
| Sow parity | 3.5 ± 1.5 |
| Total born | 15.2 ± 3.3 |
| Liveborn | 13.8 ± 3.0 |
| Stillborn | 1.1 ± 1.2 |
| Mummies | 0.3 ± 0.4 |

TABLE 3
The UNESP composite pain scale (UPAPS) for scoring pain in piglets.

| Item | Score | Score/criterion | Links to videos |
|---|---|---|---|
| Posture | 0 | Normal (any position, apparent comfort, | youtu.be/QSosCD2SD4E |
| | | relaxed muscles) or sleeping | |
| | 1 | Changes posture, with discomfort | youtu.be/SpaWsFCrPxE |
| | 2 | Changes posture, with discomfort, and | youtu.be/VjSlsRrG8yA |
| | | protects the affected area | |
| | 3 | Quiet, tense, and back arched | youtu.be/pm4hJ5163ao |
| Interaction | 0 | Interacts with other animals; interested | youtu.be/-880STgYq2I |
| and interest | | in the surroundings or sleeping | |
| in the | 1 | Only interacts if stimulated by other | youtu.be/nXjOdwn3dyw |
| surroundings | | animals; interested in the surroundings. | |
| | 2 | Occasionally moves away from the | youtu.be/2k2JDr5U6As |
| | | other animals, but accepts approaches; | |
| | | shows little interest in the surroundings | |
| | 3 | Moves or runs away from other animals | youtu.be/se70oYXcWFw |
| | | and does not allow approaches; | |
| | | disinterested in the surroundings | |
| Activity | 0 | Moves normally or sleeping | youtu.be/cC75t7L5-YA |
| | 1 | Moves with less frequency | youtu.be/lQo9wq8LAn8 |
| | 2 | Moves constantly, restless | youtu.be/YQRJjijLvpk |
| | 3 | Reluctant to move or does not move | youtu.be/Zyx0G3Wpt8o |
| Attention to | | A. Elevates pelvic limb or alternates the | youtu.be/UD99ftO7HE0 |
| the affected | | support of the pelvic limb | |
| area | | B. Scratches or rubs the painful area | youtu.be/7idfFk1harE |
| | | C. Moves and/or runs away and/or | youtu.be/u-Pqubom278 |
| | | jumps after injury of the affected area | |
| | | D. Sits with difficulty | youtu.be/ETNEOCVV4h0 |
| | 0 | All the above behaviors are absent | |
| | 1 | Presence of one of the above behaviors | |
| | 2 | Presence of two of the above behaviors | |
| | 3 | Presence of three or all the above behaviors | |
| Miscellaneous | | A. Wags tail continuously and intensely | youtu.be/pU5dGZFNRHc |
| behaviors | | B. Bites the bars or objects | youtu.be/cF3dsq7gMtk |
| | | C. The head is below the line of the | youtu.be/ZcIgngclRpI |
| | | spinal column. | |
| | | D. Presents difficulty in overcoming | youtu.be/HlvdOI3lGuY |
| | | obstacles (example: another animal) | |

| | |
|---|---|
| 0 | All the above behaviors are absent |
| 1 | Presence of one of the above behaviors |
| 2 | Presence of two of the above behaviors |
| 3 | Presence of three or all the above behaviors |

### Example 2

Flunixin meglumine is a nonsteroidal anti-inflammatory drug approved to manage pyrexia associated with swine respiratory disease. In the United States, no analgesic drugs are approved for use in swine by the FDA, although they are needed to manage painful conditions. This study evaluated the pharmacokinetics and bioavailability of intranasal versus intramuscular flunixin in grower pigs. Six pigs received 2.2 mg/kg flunixin either intranasally or intramuscularly before receiving flunixin via the opposite route following a 5-day washout period. Plasma samples were collected over 60 hours and analyzed using ultra-performance liquid chromatography and tandem mass spectrometry to detect flunixin plasma concentrations. A non-compartmental pharmacokinetic analysis was performed. The median Cₘₐₓ was 4.0 μg/mL and 2.7 μg/mL for intramuscular and intranasal administration, respectively, while the median AUC_{inf} was 6.9 hr*μg/mL for intramuscular administration and 4.9 hr*μg/mL for intranasal administration. For both routes, the median Tₘₐₓ was 0.2 hours, and flunixin was detectable in some samples up to 60 hours post-administration. Intranasal delivery had a relative bioavailability of 88.5%. These results suggest that intranasal flunixin has similar, although variable, pharmacokinetic parameters to the intramuscular route, making it a viable route of administration for use in swine.

Flunixin meglumine (Banamine®) is a nonsteroidal anti-inflammatory drug (NSAID) approved by the United States Food and Drug Administration (U.S. FDA) for specific indications in cattle, horses, and swine. Banamine®-S, an injectable formulation of flunixin meglumine, is approved by the U.S. FDA to treat pyrexia associated with swine respiratory disease at a single intramuscular (IM) dose of 2.2 mg/kg. As a non-selective cyclooxygenase inhibitor, flunixin reduces the production of prostaglandins, which is responsible for signs of inflammation including pain, erythema, heat, swelling, and loss of function (Odensivik, 1995 and Ricciotti & FitzGerald, 2012). Various studies have demonstrated the analgesic effects of flunixin for multiple painful conditions in swine, including sow lameness, inflammatory hyperalgesia in piglets, and piglet processing (Pairis-Garcia et al., 2014, Levionnois et al., 2017, and Nixon et al., 2021), but flunixin is not labeled for analgesic use in swine despite these publications.

In the United States, no NSAIDs are approved for analgesic use in swine by the FDA. However, flunixin is commonly administered in an extralabel manner for pain associated with lameness in sows, which is a common reason for culling due to decreased growth and reproductive performance associated with the condition (Bates et al., 2014 and Pairis-Garcia et al., 2013). In many countries including the U.S., piglets undergo processing procedures shortly after birth in order to improve the efficiency and economics of the swine industry. These procedures can include tail docking to reduce tail biting and injury, the placement of ear tags for identification, clipping of needle teeth to prevent sow teat injury, and castration of males to reduce aggression, prevent unwanted reproduction, and prevent boar taint in the meat (Michigan State University Extension, 2019 and Nixon et al., 2021). These procedures have been deemed painful through measuring increased rump scratching and cortisol levels, high-frequency vocalizations, and escape attempts during and after the procedures (Viscardi and Turner, 2018 and Marchant-Forde et al., 2014). Currently, consumers have a growing concern over animal welfare, especially in the swine industry, and therefore desire that animals receive adequate pain management when needed, including during piglet processing (Kittrell et al., 2020).

Legislation in Europe and Canada requires piglets to receive analgesia during processing due to welfare concerns, but no such legislation is in place in the United States. Additionally, analgesics in the United States are not used on swine farms due to time constraints and economics associated with administering the drug (Imeah et al., 2020). Having a needle-less administration method of an analgesic would improve animal welfare, worker safety, and efficiency while decreasing disease spread within a farm and reducing injection site abscesses that can lead to carcass condemnation (Imeah et al., 2020). While there is a transdermal formulation of flunixin approved for cattle, studies have shown that it yields low blood concentrations in swine that are not expected to mitigate pain, making it a likely ineffective analgesic for this species (Kittrell et al., 2020). Meloxicam has been approved for swine pain management in Europe and Canada, but a recent study suggested that flunixin may be more effective than meloxicam in providing analgesia to swine following tail docking and castration (Nixon et al., 2021 and 2022). The pharmacokinetics of flunixin in swine has been evaluated when administered intramuscularly, intravenously, transdermally, and orally (Kittrell et al., 2020, Pairis-Garcia et al., 2013, Cramer et al., 2019; Nixon et al., 2021, Nixon et al., 2020). However, no studies have evaluated the pharmacokinetics of intranasal (IN) flunixin in pigs to determine if this route of administration would achieve adequate plasma drug concentrations for pain mitigation.

Previous studies have investigated the IN administration of various drugs in multiple species and shown promising results. For example, a study investigated the use of IN midazolam and tetrabenazine in swine and found that this route of administration provides rapid absorption and adequate plasma drug concentrations (Lacoste et al., 2000 and Arora et al., 2020). Additionally, a study in humans showed that IN ketorolac was well tolerated, effective in treating post-operative pain, and was a fast and simple route of administration (Brown et al., 2009). The objective of this study was to determine the pharmacokinetics and bioavailability of IN flunixin following a single dose of 2.2 mg/kg administered to grower pigs to determine how this drug delivery route compares to the standard intramuscular (IM) administration route.

### Materials and Methods.

This study was approved by the North Carolina State University Institutional Animal Care and Use Committee (IACUC #20-195-A). A pilot study in one animal was initially performed to compare jugular venous drug concentrations to those of other sites (femoral artery and auricular artery), as there was concern that drainage of the nasal sinus would produce higher concentrations of flunixin in the blood collected from the jugular vein.

The pilot study involved one female Yorkshire cross grower pig sourced from a production farm weighing 25.3 kg. The pig was sedated with IM Telazol®, ketamine, and xylazine (TKX). The Telazol® was reconstituted with ketamine (250 mg; 2.5 mL) and xylazine (250 mg; 2.5 mL; Dechra Veterinary Products, Overland Park, KS, USA) and administered IM at a dose of 0.03 mL/kg (1.5 mg/kg) for each medication before being intubated and maintained on isoflurane anesthesia. A femoral arterial catheter, jugular venous catheter, and auricular arterial catheter were placed using sterile technique. The pig received 3.0 mg/kg (50 mg/mL) of flunixin (Banamine®-S, Merck Animal Health, Madison, NJ, USA) intranasally. A higher dose than the label dose was chosen to ensure that the drug would be detectable since this study's purpose was to determine an appropriate catheter site. Using an intranasal mucosal atomization device (MAD) (Teleflex, Morrisville, NC, USA) with 1 mL luer lock syringe, each nostril was infused with 0.75 mL (37.5 mg) of flunixin to administer a total of 1.5 mL (75 mg). Plasma samples were collected from each catheter site at pre-treatment (blank), 0.08, 0.25, 0.5, 0.85, 1, 1.5, 2, 2.5, and 3 hours post-treatment and analyzed using ultra performance liquid chromatography and tandem mass spectrometry (UPLC-MS/MS) to determine flunixin concentrations. A non-compartmental pharmacokinetic analysis was then performed.

### Animals and Housing for Pharmacokinetic Study:

The IN and IM pharmacokinetic study was approved by the North Carolina State University Institutional Animal Care and Use Committee (IACUC #20-195). Six female Yorkshire cross grower pigs approximately 8 weeks of age with no history of flunixin administration in a 30-day period were sourced from a production farm and enrolled in the study. The pigs were determined to be healthy by veterinary physical examination and pigs initially weighed between 18.0-22.0 kg. The pigs were individually housed in climate-controlled rooms with a 12:12 light dark cycle at the North Carolina State University College of Veterinary Medicine to prevent cross contamination between pigs and damage to jugular catheters. The pens had non-slip flooring, allowed the pigs to move freely, provided enrichment, and were cleaned at least twice daily. The pigs had ad libitum access to water via a nipple drinker and were fed 0.5 quarts of Nature's Match twice daily. The pigs acclimated for 1-2 days before they were anesthetized for jugular catheter placement as described in the pilot study above and were provided approximately 24 hours to recover from anesthesia before the study began. From the time of arrival until the end of the study, the pigs were monitored at least twice daily for general appearance, attitude, appetite, and wellbeing.

To facilitate jugular catheter placement, the pigs were sedated with Telazol®, reconstituted with ketamine and xylazine as previously described, intubated, and maintained on gas isoflurane anesthesia. A 22-gauge, 10 cm intravenous catheter (MILA, International, Inc., Florence, KY, USA) was placed into the jugular vein using sterile technique. It was sutured in place with 3-0 nylon and covered in Ioban™ (3M, St. Paul, MN, USA) to prevent damage. The pigs wore custom jackets throughout the study to protect the catheters and an extension set was attached to allow for easy catheter access and restraint-free blood collection.

### Dosing and Sample Collection:

All pigs were randomized to determine the first administration route. Group 1 (n=3 pigs) and Group 2 (n=3 pigs) were used in a cross-over design and received 2.2 mg/kg flunixin (Banamine®-S, Merck Animal Health, Madison, NJ, USA) by either the IM or IN route. Following a 5-day washout period, the pigs were administered 2.2 mg/kg flunixin via the opposite route. IN administration was completed using a MAD600 MADgic laryngo-tracheal mucosal atomization device (Teleflex Medical, Morrisville, NC, USA). The luer lock syringe was attached to this device and drug was placed into the nostril. Before administration, 0.22 mL of the drug was added to the device to account for dead space volume. After the IN dose, pigs were encouraged to keep their heads raised for approximately 10 seconds to prevent leakage of the drug out of the nasal cavity. Intramuscular administration was performed in the omotransversarius muscle using a 20G needle. All pigs were euthanized at the end of the study with Fatal-Plus® solution (Vortech Pharmaceuticals, Dearborn, MI, USA) at a dose of 1 mL/4.5 kg IV and death was confirmed via cardiac auscultation and a lack of reflexes.

Blood samples were collected at 0 (pre-treatment), 0.16, 0.33, 0.5, 0.75, 1, 1.5, 2, 3, 4, 6, 8, 12, 18, 24, 36, 48, and 60 hours post treatment. To avoid heparin contamination in samples, at least 1 mL of blood was collected as a purge sample before 1-2 mL of blood was collected and placed into lithium heparin tubes (BD Vacutainer, Franklin Lakes, NJ, USA). The purge blood was injected back into the catheter before the catheter was flushed with 3-4 mL of heparinized saline to prevent coagulation. The blood tubes were placed on ice for no more than 1 hour before they were processed. Plasma was separated by centrifugation at 3500×g for 10 minutes at 4° C., equally split between two cryovials, mixed, and stored at −80° C. until analysis.

### Chemicals and Reagents:

The reagents were of LC/MS grade. Acetonitrile (ACN), methanol (MeOH), and formic acid were supplied by Fisher Chemical (Raleigh, NC, USA). Phosphoric acid was supplied by Aldrich Chemistry (Burlington, MA, USA). The flunixin meglumine reference standard was purchased from Sigma-Aldrich (St. Louis, MO, USA). The ultrapure water was supplied by Waters Corporation (Milford, MA, USA). Analytical analysis of flunixin was carried out via ultra-performance liquid chromatography (UPLC) and tandem mass spectrometric (MS/MS) detection (Waters Corporation, Milford, MA, USA). The UPLC-MS/MS system consisted of a Acquity UPLC I class Binary Solvent Manager, Acquity UPLC sample Manager FTN and a Xevo TQD tandem mass spectrometer (Waters Corporation, Milford, MA, USA).

### Sample Preparation:

To prepare samples, 100 μL of plasma was pipetted into a clean borosilicate glass tube (disposable culture tubes, CWR). To pretreat the plasma, 500 μL of 4% phosphoric acid prepared in water was added to each tube. Solid phase extraction was performed on an oasis prime HLB 96 well μElution plate (Waters Corporation, Milford, MA, USA). The plate was prepared by conditioning the plate with 500 μL of methanol followed by 500 μL of ultrapure water. After, 600 μL of pretreated plasma was loaded on the HLB μElution plate and passed through the plate under a vacuum (<5 mm). The pressure of the vacuum was increased as necessary to pull samples through the plate and the plate was then washed with 600 μL of 5% methanol prepared in water under a vacuum. The analyte was eluted into a clean 96 well sample plate (700 μL round 96 well samples plate, Waters Corporation, Milford, MA, USA) by a vacuum with the addition of 50 μL of an elution solution (70:30:ACN:MeOH). Then, 50 μL of ultrapure water was added to eluent and mixed thoroughly. Standards from 0.0001-0.1 μg/mL and 0.05-5 μg/mL were prepared using blank piglet plasma, which was injected with every batch. Any plasma samples with drug concentrations exceeding 5 μg/mL were diluted with blank piglet plasma prior to sample preparation and reanalyzed.

### UPLC-MS/MS Conditions:

Chromatographic separation was performed by a gradient elution on the ACQUITY UPLC BEH phenyl 1.7 μm column (2.1×100 mm) with VanGuard pre-column (Waters Corporation, Milford, MA, USA). The mobile phase solvents were 0.1% formic acid in water (A) and 0.1% formic acid in ACN (B) at a flow rate 0.4 mL/min for 5 minutes. The gradient program mobile phase conditions were 70% of A and 30% of B for the first 2.5 minutes, then changed linearly to 10% of A and 90% of B from 2.5-3.5 minutes, then immediately back to 70% of A and 30% of B from 3.5-5 minutes to re-equilibrate at the initial conditions. The column temperature was 35° C. and the autosampler temperature was maintained at 25° C. The injection volume was 5 μL for the standard curve (0.0001-0.1 μg/mL) and samples of this range. The injection volume was 0.3 μL for standard curves (0.05-5 μg/mL) and samples of this range. The positive electrospray ionization (ESI (+)) was used with the multiple reactions monitoring (MRM). The true page source voltages were 0.4 kV and 50 V for the capillary and cone, respectively. The source desolvation temperature was 550° C. The source desolvation gas flow was 1000 L/hr and the cone gas was 50 L/hr. The MS file cone voltage setting was 44 V with collision energy setting of 30 V. Argon was used as the collision gas and nitrogen was used as the desolvation and cone gasses. Quantification was performed using the transition Parent (m/z): 297.05 and Daughter (m/z): 264.03 with retention time 2.41 minutes.

### Calibration Curve:

The calibration curve of flunixin was fitted with a weighted (1/concentration) linear equation. The calibration ranges of 0.0001-0.1 μg/mL and 0.05-5 μg/mL were linear with a coefficient of determination, R², greater than or equal to 0.99. Each calibration standard concentration could be back calculated to within 15% of the true concentration.

### Precision and Accuracy:

A total of 6 replicates at low, medium, and high concentrations of flunixin (0.003, 0.015 and 0.07 μg/mL for calibration curve of 0.001-0.1 μg/mL and 0.3, 0.7, 1.5 and 3 μg/mL for calibration curve of 0.05-5 μg/mL) were tested over 3 days. The inter-day and intra-day precision and accuracy were calculated. The precision was 1.2-5.1% and recovery was 96.0-110.0%. The limit of quantification was determined based on visual inspection of the chromatographs, inter-day precision (<15%) and accuracy (85-115%) and signal-to-noise ratio 5 times the blank piglet plasma. The limit of detection was determined based on chromatograph and signal-to-noise ratio 3 times the blank piglet plasma. The limit of quantification and limit of detection was recognized as 0.001 μg/mL and 0.0001 μg/mL for the lower standard curve (0.0001-0.1 μg/mL) and 0.1 μg/mL and 0.05 μg/mL for higher standard curve (0.05-5 μg/mL), respectively.

### Pharmacokinetic Analysis:

A non-compartmental pharmacokinetic analysis of flunixin in plasma was performed using commercially available software (Phoenix WinNonlin™, version 8.3, Certara, St. Louis, MO, USA). The pharmacokinetic parameters were estimated for flunixin in plasma after IN and IM administration included the elimination rate constant (λz), terminal half-life (HL λz), the time to maximum concentration (Tₘₐₓ), the maximum plasma concentration (Cmax), the area under the curve from time zero to the last time point (AUCₗₐₛₜ), the area under the curve from time zero to infinity (AUC_{inf}), the apparent volume of distribution per fraction absorbed (Vz/F), the apparent clearance per fraction absorbed (Cl/F), and the AUC portion extrapolated (AUCₑₓₜᵣₐₚ). These values were estimated using the linear log trapezoidal method. The relative bioavailability was calculated using the equation below:

$$Relative bioavailability IN ( \% ) = ( {AUC}_{inf} IN / {AUC}_{inf} IM ) \times ( Dose IM / Dose IN ) \times 100.$$

### Statistical Analysis:

Parameters (AUC_{inf}, half-life, Cₘₐₓ, Tₘₐₓ, and MRT) for each route of administration were compared with a Wilcoxon Signed Rank Test using JMP® Pro Software version 16.0 (SAS, Cary, NC, USA). Differences were considered significant at p<0.05.

### Results:

### Observations.

No major adverse effects on the pigs were observed following IN or IM administration of flunixin. A limited gross necropsy of the nasal cavity following euthanasia revealed no negative effects to the nasal cavity following IN administration.

### Pilot Study Results:

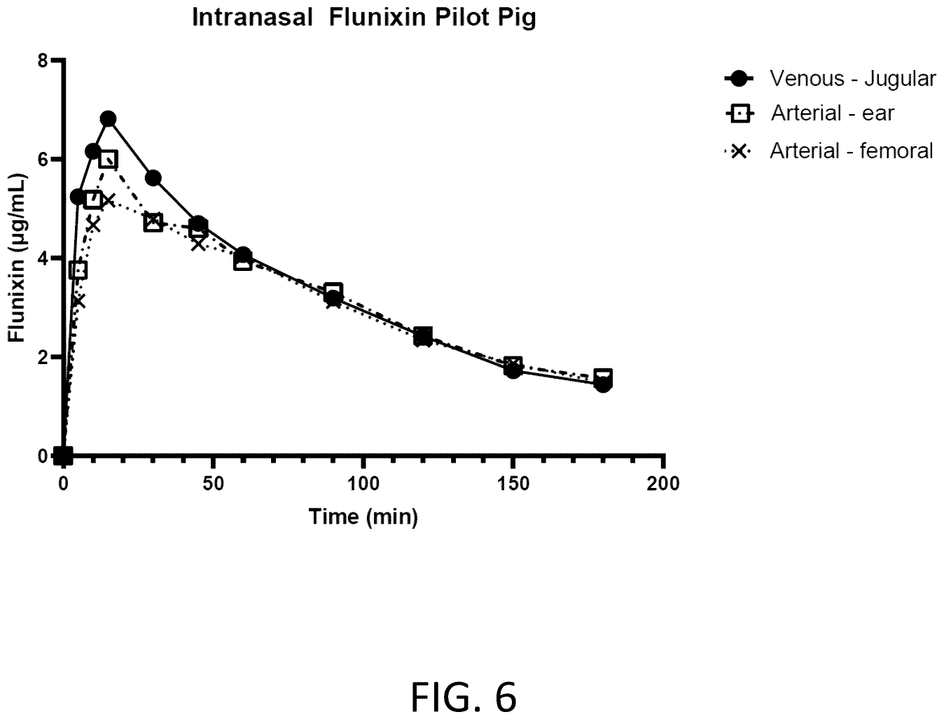

FIG. 6 shows the plasma concentration of flunixin from each catheter site following the administration of 3.0 mg/kg IN flunixin. In the pilot study, the jugular vein did not yield subjectively elevated flunixin concentrations in the plasma compared to the femoral artery and auricular artery, although data is available from only one animal. From 0.08-0.5 hours, drug concentrations from the jugular vein were slightly higher than the other sites, but beginning at 0.75 hours, the drug concentrations were very similar between all catheter sites. The time to peak concentration (Tₘₐₓ) was 0.25 hours for all sites. The peak concentration (Cₘₐₓ) was 6.82 μg/mL for the jugular vein, 6 μg/mL for the auricular artery, and 5.17 μg/mL for the femoral artery. The area under the curve (AUC) for the jugular vein, the auricular artery and the femoral artery was 769.1, 767.9, and 737.6 min*μg/mL, respectively. Thus, for ease of catheter maintenance and minimally invasive sampling, the jugular catheter site was elected for the full pharmacokinetic study.

### Intramuscular and Intranasal Delivery Results of Pharmacokinetic Study:

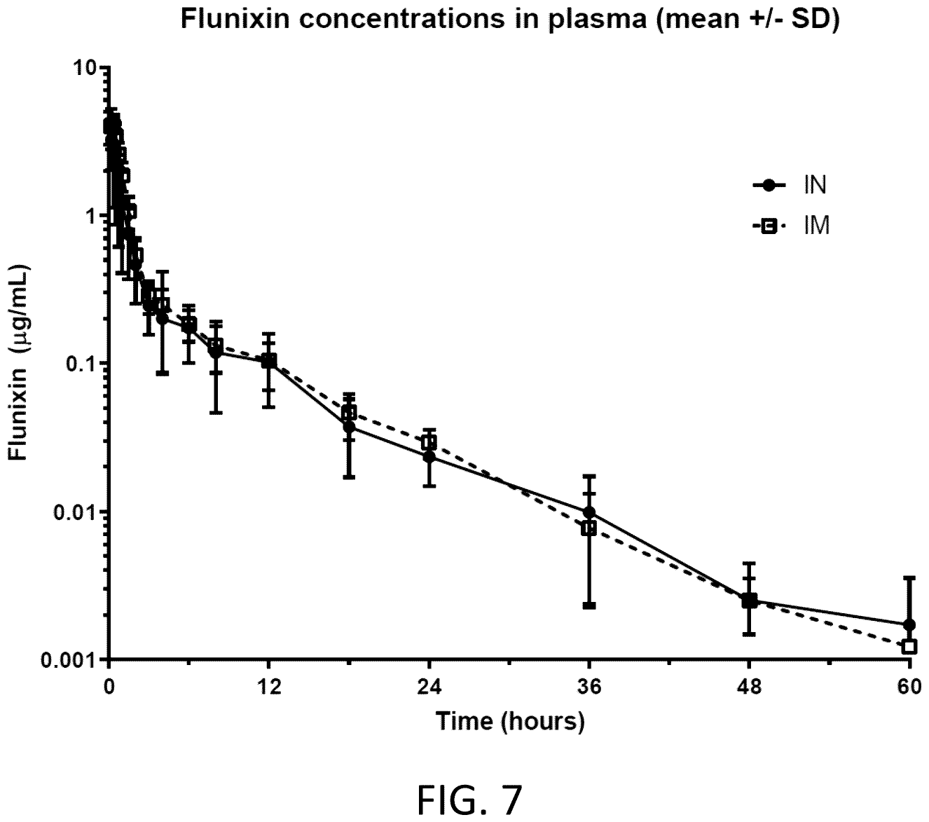

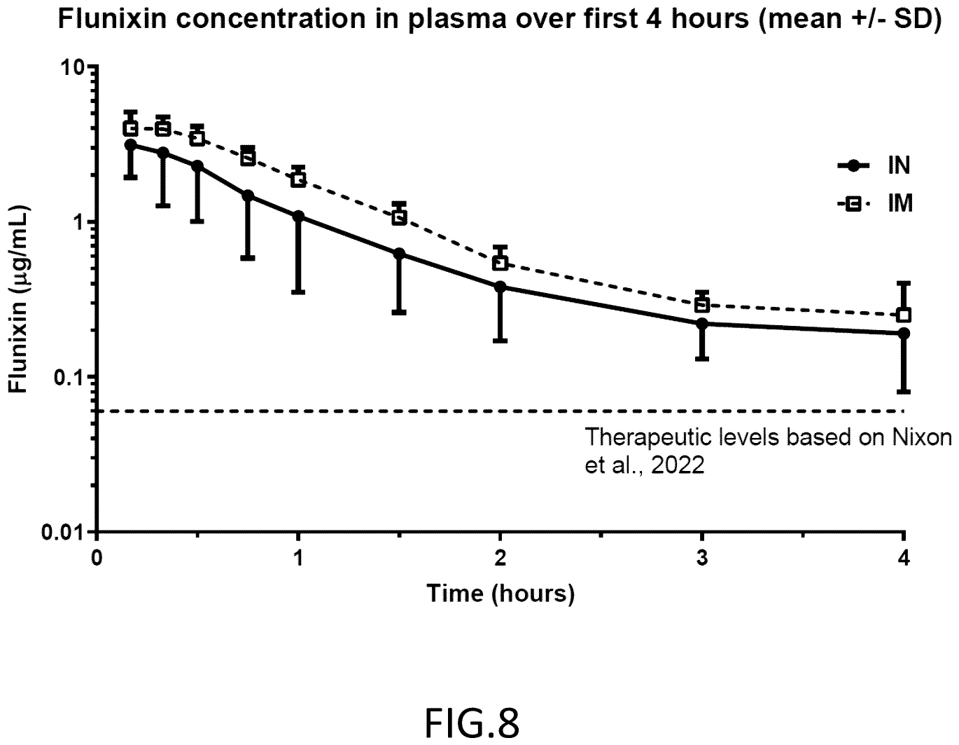

FIG. 7 shows the mean plasma concentrations ±standard deviation (μg/mL) of flunixin following IM and IN administration of 2.2 mg/kg flunixin meglumine over 60 hours. Following both IN and IM administration, flunixin was detectable in plasma in some pigs up to 60 hours post-administration. FIG. 8 shows the mean±standard deviation flunixin plasma concentration (μg/mL) during the first 4 hours of administration, revealing that both IM and IN flunixin do reach concentrations above the therapeutic level (0.06 μg/mL) of the drug (Nixon et al., 2022). The results of pharmacokinetic parameters for both IM and IN administration are shown in Table 1 as median (range). The relative bioavailability of IN administration compared to IM was 88.5 (39.1-113.8) %. Table 2 compares the AUC_{inf} of IM and IN administration in each individual pig, along with relative bioavailability of IN administration in each pig.

The Cₘₐₓ was the only parameter found to be significantly greater for the IM route compared to the IN route (p=0.047). Neither the Tₘₐₓ was significant with p=0.500, nor was the AUC (p=0.078). Terminal half-life was not significant with a p value=0.578.

Our study is the first to report the pharmacokinetic parameters of IN flunixin in grower pigs at a dose of 2.2 mg/kg. We chose to study flunixin based on previous research that compared the pharmacokinetics of IM flunixin, meloxicam, and ketoprofen in swine (Nixon et al., 2021). This study showed that flunixin (2.2 mg/kg) had a longer terminal half-life, and higher bioavailability compared to ketoprofen (3 mg/kg) and meloxicam (0.4 mg/kg), indicating that it may have greater tissue penetration and have the most potential for analgesic use (Nixon et al., 2021). In addition, a pharmacodynamic study found flunixin to be superior to other NSAIDs for mitigating pain and stress associated with castration and tail docking (Nixon et al., 2021 and 2022). However, there is an unmet need for minimally invasive and needleless drug delivery options for analgesics on swine farms, thus the impetus for the present study.

Our study found a good relative bioavailability (88.5%) and identical Tₘₐₓ (0.2 hours) for the IN group compared to the IM group, revealing that IN flunixin reaches similar plasma concentrations in the same amount of time as IM flunixin. The rich blood supply to the nasal mucosa likely allows for rapid absorption of flunixin with minimal swallowing or risk of first pass hepatic metabolism (Hampton et al., 2021 and Enomoto et al., 2022). In order to minimize loss of the drug from the nose, a commercially-available laryngo-tracheal mucosal atomization device (MAD600 MADgic, Teleflex Medical, Morrisville, NC, USA) was used to administer the flunixin intranasally. The MAD atomizer is designed to create a fine mist of particles and ease the absorption of the drug through mucosa, potentially increasing bioavailability, although future research is needed to determine if atomization is truly necessary. A small volume of drug is suitable to be administered by the smaller size of MAD atomizer (1 mL or 3 mL) and decreases the chance of swallowing the drug or spillage from the administration site (Santangelo et al., 2019 and Enomoto et al., 2022). Administering IN flunixin to smaller piglets—the target age group for this route of administration—might be also easier than grower pig because it is easier to restrain them. Therefore, IN flunixin administered using an atomizer for younger, smaller piglets may have higher relative bioavailability compared to that in our study, but this needs to be investigated further.

In our study, 2.2 mg/kg IN flunixin had a median Cₘₐₓ of 2.7 μg/mL, but was lower than that of IM administration (4.0 μg/mL). Both route had similar ranges in plasma concentrations, and varied by over 2-fold in concentrations. While the IN route did not reliably reach the same plasma concentrations as the IM route, it was still well absorbed while being a painless route of delivery since the use of a needle was not required. More importantly, the AUC was similar between the two routes, indicating that the overall exposure to flunixin is sufficient following IN administration. Both routes of administration had a geometric mean Tₘₐₓ of 0.2 hours, showing that the drug was absorbed quickly, which is a useful characteristic of an analgesic being used during acute pain, such as processing procedures. Intranasal flunixin had a median half-life of 7.4 hours, which was almost identical to that of IM flunixin (7.3 hours). Using estimates of 10 half-lives to eliminate 99.99% of a dose administered, one would not expect any flunixin residues by the time a young animal was sent to slaughter, although tissue residues would need to be performed to confirm this estimate.

A study by Kittrell et al. (2020) evaluated extralabel administration routes of flunixin in piglets, including IV (2.2 mg/kg), IM (2.2 mg/kg), PO (3.3 mg/kg), and TD (3.3 mg/kg). Compared to these results, our IN administration has a higher mean Cₘₐₓ (2.7 μg/mL) compared to TD administration (0.04 μg/mL) but lower than that of PO administration when a higher dose was used (4.99 μg/mL). IV administration did have a much higher maximum observed drug concentration (12.03 μg/mL). However, IV administration of drugs in pigs is difficult and requires advanced technical skill and the use of a snare, which is stressful to the animals. Oral administration is challenging since there is no way to ensure proper dose administration to individual pigs when provided in the feed. IN administration has a lower mean Tₘₐₓ (0.2 hours) compared to PO administration (0.93 hours) and TD administration (22.50 hours), showing that IN flunixin was quickly absorbed compared to other methods, making it ideal for use during acute pain. The median half-life of IN flunixin (7.4 hours) is comparable to IV administration (7.06 hours), but shorter than PO administration (11.38 hours) and TD administration (38.89 hours), which indicated that IN flunixin was eliminated quickly compared to PO and TD flunixin. Although the tissue half-life of IN flunixin needs to be investigated to estimate the meat withdrawal interval, the shorter plasma half-life of IN flunixin may predict shorter tissue half-life, which might be helpful in older sows experiencing lameness who may soon go to slaughter (Bates et al., 2020). The label withdrawal time for IM flunixin in pigs is 12 days. Our data suggests that the withdrawal time for the IN route may be comparable, but unless approved by the FDA via this route, an extended withdrawal interval would still apply.

There was considerable variability observed in the plasma concentrations, pharmacokinetic parameters, and bioavailability of IN flunixin compared to IM flunixin in individual pigs. Many factors, including nasal tissue pH, presence of mucus, nasal anatomy and blood flow, drug administration technique, animal individuality, and drug loss posteriorly into the oropharynx can influence drug absorption through the nasal mucosa (Enomoto et al., 2022). In our study, the drug was administered intranasally to all of the pigs by the same investigator to minimize the variability of drug administration techniques. The variables associated with IN administration may indicate the need for a dose adjustment to be considered and a need for a device that can ensure proper administration of the drug while preventing leakage. While the pigs in our study had similar genetics, they were not from a homogenous population, which could further contribute to the variability seen between individuals.

It is important to note limitations of our study, including a small sample size (n=1 for pilot study and n=6 for the cross-over PK study) and the utilization of individual housing compared to the group housing that would occur on production farms. The jugular vein was selected for sampling in pharmacokinetic cross-over study to ease the maintenance of catheter throughout the study period based on pilot study data, thus a possibility of overestimation of drug concentration in intranasal group was considered. However, because the pilot study showed similar AUCs over the study period, the jugular site was deemed to acceptable. Individual housing in a controlled environment was performed to allow for fewer variables and more consistent results. Additionally, the pigs used in this study were healthy, but pharmacokinetic parameters could be influenced by illness, stress, and pain (Bates et al., 2020). Future research should be conducted on commercial production farms to resemble more realistic clinical use and to obtain a larger sample size. Finally, our ideal target population for IN flunixin administration is newborn piglets, but we studied the use of flunixin in grower pigs in order to obtain full pharmacokinetic profiles while limiting the number of animals used. Future research should repeat this study in piglets <10 days of age to determine if the IN pharmacokinetics differ across ages, as well to observe for any adverse drug effects. However, challenges associated with this include increased feeding intervals and a less developed immune system in piglets. We anticipate that piglets will be much easier to restrain for IN administration of flunixin compared to grower pigs. As discussed previously, since sow lameness is prevalent in the industry, IN flunixin administration should also be studied in sows for the same reasons.

An intranasal delivery method of flunixin would be a vital addition to the swine industry by significantly improving animal welfare through providing fast and effective pain management during painful procedures such as piglet processing. It would also improve carcass quality by avoiding muscle damage caused by an injection and improve worker safety by minimizing the use of needles. However, a formal meat withdrawal interval needs to be established to safely use this drug in pigs to avoid drug residue contamination in the food supply. Further research is required to determine the analgesic efficacy of intranasal flunixin in both piglets and sows to investigate the use of this drug in various age groups and confirm the most appropriate dose.

Intranasal flunixin resulted in similar, but highly variable, plasma concentrations as compared to IM flunixin and were within the therapeutic range for analgesia associated with castration and tail docking in piglets in healthy grower pigs.

### Tables:

TABLE 1
Plasma pharmacokinetic parameters after intramuscular
and intranasal administration of 2.2 mg/kg
flunixin meglumine in six grower pigs.

| | Intramuscular | | | |
|---|---|---|---|---|
| Parameters | administration | | Intranasal administration | |
| λz (1/hr) | 0.1 | (0.07-0.13) | 0.09 | (0.07-0.13) |
| HLλz (hr) | 7.3 | (5.3-9.6) | 7.4 | (5.3-9.5) |
| Tₘₐₓ (hr) | 0.2 | (0.2-0.3) | 0.2 | (0.2-0.3) |
| Cₘₐₓ (μg/mL) | 4.0 | (2.9-5.8) | 2.7 | (2.1-5.6) |
| AUClast (hr* μg/mL) | 6.9 | (5.0-8.5) | 4.8 | (3.0-8.9) |
| AUC_{inf} (hr* μg/mL) | 6.9 | (5.0-8.5) | 4.9 | (3.0-8.9) |
| AUC Extrap (%) | 0.3 | (0.2-0.5) | 0.5 | (0.1-0.8) |
| Relative bioavailability (%) | | | 88.5 | (39.1-113.8) |
| CL/F (L/hr/kg) | 0.3 | (0.3-0.4) | 0.5 | (0.2-0.7) |
| Vz/F (L/kg) | 3.2 | (2.7-5.8) | 5.4 | (1.9-8.3) |

λz: elimination rate constant, HL_{λz}: terminal half-life, Tₘₐₓ: time to the maximum concentration, Cₘₐₓ: maximum concentration, AUCₗₐₛₜ: area under the curve from time zero to the last time point, AUC_{inf}: area under the curve from time zero to infinity, AUCₑₓₜᵣₐₚ: extrapolation of AUC, Cl/F: clearance per fraction absorbed, V/F: volume of distribution per fraction absorbed.

TABLE 2
Area under the curve from time zero to infinity (AUC_{inf}) and relative
bioavailability of intranasal (IN) administration of flunixin
(2.2 mg/kg) compared to intramuscular (IM) administration of
flunixin (2.2 mg/kg) in individual grower pig (n = 6)

| | AUC_{inf} of IM | AUC_{inf} of IN | Relative |
|---|---|---|---|
| Pig # | administration | administration | bioavailability (%) |
| 1 | 4.99 | 4.74 | 95.06 |
| 2 | 7.63 | 2.98 | 39.11 |
| 3 | 6.25 | 2.98 | 47.63 |
| 4 | 7.82 | 8.90 | 113.78 |
| 5 | 8.54 | 7.07 | 82.84 |
| 6 | 5.29 | 4.98 | 94.22 |

### Example 3

The objective of this study was to evaluate the effects of intranasal flunixin and intra-inguinal lidocaine application on the castration associated inflammatory response in piglets, as described in Example 1.

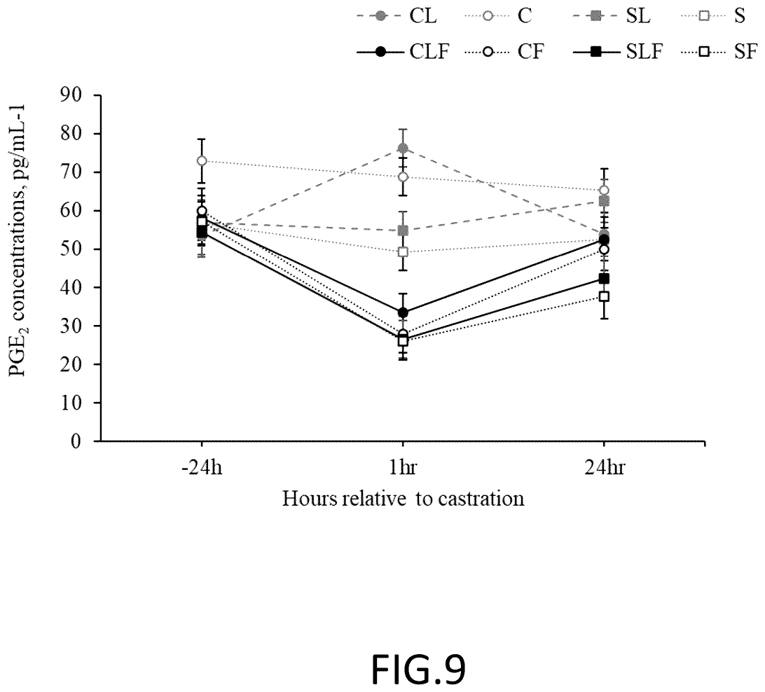

Castrated and non-castrated piglets that received flunixin intranasally had lower serum PGE₂ concentrations at 1 h post-castration than piglets that did not receive the drug (FIG. 9). Even though flunixin decreased PGE₂ immediately after castration in this study, at 24 h post castration there were no observed differences from the other treatments. Intra inguinal lidocaine did not mitigate inflammation at any time point. While buffering lidocaine may have lowered pain associated with the substance injection, possible damage to the spermatic cord during injection might have been responsible to the increased inflammation observed in this study, as previously suggested (Lopez-Soriano et al., 2023). Another explanation is that lidocaine may directly increase PGE₂.

Results from the current study suggest that intranasal flunixin is effective in mitigating inflammatory response of piglets undergoing castration as determined by decreased PGE₂ levels immediately following the procedure. However, a single intranasal flunixin administration was not effective in mitigating long-term post-operative inflammation. Administration frequency and dosing of intranasal flunixin may be modified to control pain for a longer period post-castration.

What is claimed is:

1. A method of treating and/or reducing pain in an animal, the method comprising administering at least one dose of a composition comprising an analgesic to the animal, wherein the at least one dose is administered intranasally.

2. The method of claim 1, wherein at least one dose is administered to the animal prior to, concomitant with, or after performing a procedure on the animal, and wherein the administration reduces pain in the animal caused by the procedure.

3. The method of claim 2, wherein the procedure comprises one or more of castration, dehorning/disbudding, ear notching, branding, teeth clipping, tail docking, and other surgical procedures associated with tissue damage.

4. The method of claim 3, wherein the pain reduction in the animal is associated with one or more of: a reduction in cortisol levels, a reduction in prostaglandin E2 (PGE₂), and/or a change in activity or behavior of the animal.

5. The method of claim 1, wherein the at least one dose is administered to the animal to treat pain.

6. The method of claim 5, wherein the pain is caused by a surgical procedure, a processing procedure, an injury, an inflammatory condition, a respiratory disease, and/or an infection.

7. The method of claim 1, wherein the method further comprises administering at least one second dose of an analgesic.

8. The method of claim 7, wherein the at least one second dose is administered intranasally.

9. The method of claim 7, wherein the at least one second dose is administered intramuscularly, intravenously, transdermally, or orally.

10. The method of claim 1, wherein the intranasal administration comprises use of a spraying, nebulizing, or atomizing device.

11. The method of claim 1, wherein the analgesic is a non-steroidal anti-inflammatory drug (NSAID).

12. The method of claim 11, wherein the NSAID is selected from the group consisting of flunixin, ketoprofen, and meloxicam, or a pharmaceutically acceptable salt thereof.

13. The method of claim 11, wherein the NSAID is flunixin, or a pharmaceutically acceptable salt thereof.

14. The method of claim 1, wherein the composition is administered at a dose ranging from about 0.1 mg/kg to about 10 mg/kg of the analgesic.

15. The method of claim 1, wherein the composition is administered at a dose ranging from about 1 mg/kg to about 5 mg/kg of the analgesic.

16. The method of claim 1, wherein the animal is selected from the group consisting of horses, cows, pigs, sheep, goats, cats, and dogs.

17. The method of any of claim 1, wherein the animal is a pig.

18. The method of claim 17, wherein the pig is a neonatal pig.

19. The method of claim 18, wherein the pig is at least 3 days old.

20. The method of claim 1, wherein the animal is a cow.
